(19) United States
(12) Patent Application Publication
(10) Pub. No.: US 2024/0076712 A1
(43) Pub. Date: Mar. 7, 2024

(54) COMPOSITIONS AND METHODS FOR INSTANT NUCLEIC ACID DETECTION

(71) Applicant: Wuhan University, Wuhan (CN)

(72) Inventors: Hao Yin, Wuhan (CN); Shuhan Lu, Wuhan (CN); Ying Zhang, Wuhan (CN); Xiaohan Tong, Wuhan (CN); Kun Zhang, Wuhan (CN); Xi Zhou, Wuhan (CN); Dingyu Zhang, Wuhan (CN)

(21) Appl. No.: 18/271,654

(22) PCT Filed: Jan. 7, 2022
(86) PCT No.: PCT/CN2022/070850

§ 371 Date: Jul. 10, 2023

(30) Foreign Application Priority Data
Jan. 8, 2021 (WO) PCT/CN2021/070963

(51) Int. Cl. C12Q 1/44; C12N 9/22; C12Q 1/686

(52) U.S. Cl. CPC C12Q 1/44; C12N 9/22; C12Q 1/686; C12N 2310/20

(57) ABSTRACT

Compositions and methods are provided for simple, instrument-free and sensitive methods that enable rapid, point-of-care detection of nucleic acid molecules of interest. This is based on a surprising discovery that the relative efficiencies of amplification and CRISPR-based cleavage and detection can be tuned to favor amplification until sufficient amplified products are generated to enable detection. Example approaches include design of guide RNA and primers to target nonoptimal PAM sequences, or sequence-engineering Cas nucleases to reduce activities informing a ribonucleoprotein with the guide RNA or binding to or cleaving the substrate nucleic acid.

### BACKGROUND

In the past decades, there have been a number of large-scale outbreaks of epidemic diseases caused by viruses such as severe acute respiratory syndrome coronavirus (SARS), middle east respiratory syndrome coronavirus (MERS), human immunodeficiency virus (HIV), Zika virus, Ebola virus and the current pandemic outbreak caused by SARS-CoV-2. As of now, the world is facing a huge challenge to control the spread of SARS-CoV-2 which has caused more than many million deaths and city to city lockdown. The global spread of SARS-CoV-2 is fast, in part due to high prevalence of pre-symptomatic and asymptomatic transmission.

The limited capacity of nucleic acid diagnostic tests makes it difficult to slow the spread, as quantitative reverse transcription polymerase chain reaction (RT-qPCR)-based tests, the gold-standard for SARS-CoV-2 diagnosis, require skilled personnel, equipment infrastructure and long sample-to-answer time. A point-of-care nucleic acid testing that is sensitive to detect asymptomatic carriers and has a turnaround time fast enough to get results before gatherings is critical to reopen schools and business safely. In contrast to qPCR, isothermal amplification assays such as recombinase polymerase amplification (RPA) and loop-mediated isothermal amplification (LAMP) provide a rapid, instrument independent and low-cost alternative. However, the nonspecific amplification of these assays results in high false positive rates.

CRISPR/Cas system (clustered regularly interspaced short palindromic repeats and CRISPR-associated proteins), a RNA-guided endonuclease has been harnessed to powerful genome editing tools. Cas12a, Cas12b and Cas13a have been repurposed as promising diagnostic tools owing to their collateral degradation of ssDNA or ssRNA. Amplification of target sequences and sequentially cleavage by Cas12 or Cas13 allows detection of pathogen such as Zika virus and HPV at similar detection limit as qPCR.

These approaches include the original two-step Specific High Sensitivity Enzymatic Reporter Unlocking (SHERLOCK) and DNA Endonuclease-Targeted CRISPR Trans Reporter (DETECTR). Recently, both two-step SHERLOCK and DETECTR have been clinically validated to detect SARS-CoV-2 with high sensitivity and reliability. However, the operation of two-step method is complicated and time-consuming, and the frequent lid opening can increase the risk of contamination. STOP (SHERLOCK testing in one pot) and SHINE (SHERLOCK and HUDSON Integration to Navigate Epidemics) which only need to drop the simply extracted nucleic acid into a reaction mixture containing isothermal amplification and cleavage volume and then read out by fluorescence reader or strips. However, these assays usually require a total reaction time of approximate an hour. In contrast, Abbott's ID NOW COVID-19 test applying isothermal amplification of unextracted samples is able to report results less than 15 minutes, but it has considerable false-positive and false-negative rates.

### SUMMARY

The instant inventors have developed a nucleic acid detection assay that is one-step, fast, sensitive, reliable and flexible. This assay showed comparable detection limit to quantitative PCR (qPCR) but with significant shorter time, e.g., from 15 to 20 minutes. The instant application, therefore, provides simple, instrument-free and sensitive alternative to gold-standard qPCR, and enables rapid, point-of-care screening for nucleic acid molecules of interest.

One embodiment of the disclosure provides a method for detecting a target polynucleotide, comprising incubating the target polynucleotide in a mixture that comprises (a) a polymerase, (b) deoxynucleoside triphosphates (dNTPs), (c) primers for amplifying the target polynucleotide, (c) a CRISPR-associated (Cas) nuclease, and (d) a guide RNA comprising a spacer fragment complementary to a target fragment on the target polynucleotide, under conditions so that the polymerase effectively amplifies the target polynucleotide while the Cas nuclease is capable of cleaving the amplified target polynucleotide.

Also provided is a kit or package for detecting a target polynucleotide, comprising (a) a polymerase, (b) deoxynucleoside triphosphates (dNTPs), (c) primers for amplifying the target polynucleotide, (c) a CRISPR-associated (Cas) nuclease, and (d) a guide RNA comprising a spacer fragment complementary to a target fragment on the target polynucleotide, wherein the polymerase can effectively amplify the target polynucleotide while the Cas nuclease is capable of cleaving the amplified target polynucleotide.

Also provided is a kit or package for detecting a target polynucleotide, comprising (a) a polymerase, (b) deoxynucleoside triphosphates (dNTPs), and (c) primers for amplifying the target polynucleotide, wherein at least one of the primers includes a suboptimal PAM sequence for a Cas nuclease, or wherein the DNA fragment amplified out by the polymerase contains one or more suboptimal PAMs which are targeted by a Cas nuclease, or wherein at least of the dNTP or primers is modified to reduce cleavage or binding by a Cas nuclease.

Still further provided, in another embodiment, is a kit or package for cleaving a target polynucleotide, comprising (a) a CRISPR-associated (Cas) nuclease, and (b) a guide RNA comprising a spacer fragment complementary to a target fragment on the target polynucleotide, wherein the guide RNA, as compared to a standard guide RNA, has reduced binding to or cleaving of the target polynucleotide.

Also provided, in another embodiment, is a mutant Cas nuclease having (a) reduced activity in forming a ribonucleoprotein (RNP), (b) changed conformation, (c) reduced activity in interacting with a target PAM sequence, or (d) reduced binding to a target polynucleotide to be cleaved.

### BRIEF DESCRIPTION OF THE DRAWINGS

FIG. 1. Suboptimal PAMs mediated a faster one-pot reaction than canonical PAMs. (a-d) The fluorescence signal of Orflab gene spacer 4 and spacer 5 in collateral activity tests (a-b) and one-pot reactions (c-d) at 37° C. Suboptimal PAMs for Orflab spacer 4 (GTTG) and spacer 5 (CTTA) were mutated to canonical PAMs for spacer 4 (TTTG) and spacer 5 (TTTA), respectively. (e-h) Summary map of fluorescent kinetics for position 1-3 point-mutated suboptimal PAMs and three canonical PAMs in collateral activity test (e & f) and the corresponding one-pot reaction of spacer 4 (g) and spacer 5 (h). Time to half-maximum fluorescence was determined. Fluorescence values were determined at 40 and 20 minutes for collateral activities and one-pot reactions, respectively. (n=3). The concentrations of dsDNA substrates were 3.5 nM in collateral activity tests and 2340 fM in one-pot reactions.

FIG. 2. Sensitivity and reliability of suboptimal PAMs-mediated one-pot reactions. The sensitivity and reliability of one-pot reactions using suboptimal PAMs and canonical PAMs were compared. crRNAs targeting the Orflab gene (spacers 4 and 5) and envelope (E) gene (spacer 8) of SARS-CoV-2 were used. (a-c) The sensitivity (a-b) and reliability (c) of spacer 4 using suboptimal PAM and canonical PAM. (d-f) The sensitivity (d-e) and reliability (f) of spacer 5 using suboptimal PAM and canonical PAM. (g-i) The sensitivity (g-h) and reliability (i) of spacer 8 using suboptimal PAM and canonical PAM. The substrate concentrations of c, f and i were 2340 fM, 2340 fM and 325.5 fM respectively. The fluorescence values in c, f and i were determined 50 minutes after incubation, and the data are from ten experiments with two replicates for each experiment. For a, b, d, e, g & h, each experiment was repeated three times, and one representative result is shown in the figure. For a-i, the reaction temperature was 37° C.

FIG. 3. Competition of RPA and crRNA/Cas12a RNP cleavage in one-pot reactions. (a-b) The accumulation of RPA amplicons in one-pot reactions. Components of RPA, the concentrations of 33 nM crRNA/Cas12a RNP and 2340 fM dsDNA substrates were incubated at 37° C. for 0, 2, 4, 6, 8, 10, 12, 14, 16, 18 or 20 minutes, and the resulting RPA amplicons were analyzed in agarose gels. Arrows indicate amplicon products. (c-d) Amplification and consumption of amplicons in one-pot reactions. Each concentration of 1170 fM suboptimal and canonical PAM substrates were mixed at a ratio of 1:1 in one-pot reactions at 37° C., and the percentage of each at 0, 1, 3, 5, 7, 10, 15, and 20 minutes was determined by deep sequencing. n=5 for each time point. (e-f) In vitro cleavage activities of crRNA 4 targeting substrates with suboptimal (GTTG) or canonical (TTTG) PAM and of crRNA 5 targeting substrates with suboptimal (CTTA) or canonical (TTTA) PAM. The concentration of 50 nM crRNA/LbCas12a complex was incubated with dsDNA substrate (6 nM for spacer 4 and 10 nM for spacer 5) at 37° C. for 0, 0.5, 1, 2, 5, 10, 15, or 20 minutes to determine the cis-cleavage activity. S, substrate; P, product. (g-h) The binding affinity of RNP for suboptimal- and canonical-PAM dsDNA was determined. 0, 12.5, 25, 50, 100, 200, and 400 nM crRNA/deactivated LbCas12a (dCas12a) complexes were incubated with 5 nM dsDNA at 37° C. for 20 minutes, and EMSAs were performed to determine the bound and unbound portions. Each experiment was repeated three times, and one representative is shown in the figure.

FIG. 4. Cis-cleavage activities of 120 PAMs of four targets. (a) Correlation of one-pot reaction and cis-cleavage of 120 PAMs. Black dots represent canonical PAMs, red dots represent better performed suboptimal PAMs and blue dots represent worse performed suboptimal PAMs. The unit of one-pot reaction (X axis) is defined as time to half-maximum fluorescence (min)*an adjusted ratio based on plateau signal of each PAM. This ratio is the value of highest plateau fluorescence among 120 PAMs divided by the plateau fluorescence value of each PAM. The three suboptimal PAMs out of 30 min range in X axis still outperformed their corresponding canonical PAMs. (b) Competitive schematic workflow of amplification and cleavage in one-pot reactions. For substrates containing canonical PAM, cleavage is predominant in the initial stage of the reaction, resulting in excessive consumption of the dsDNA activator. In contrast, as amplification outcompetes cleavage for suboptimal PAM substrates, amplicons accumulate to stimulate faster and stronger fluorescence signal production.

FIG. 5. HCMV detection by suboptimal PAM-mediated one-pot reaction. (a-d) The sensitivity of suboptimal PAM-mediated one-pot reaction and qPCR assay targeting the UL55 gene of HCMV was compared. (a-b) The PUC57-UL55 plasmid was used as substrate. (c-d) The presence of HCMV virus was determined. The reaction volume of qPCR in a, c is 20 μL and the reaction volume of one-pot reaction in b, d is 30 μL, the number of copies input in two reactions were the same. (e) Schematic of detection under portable UV light and using a lateral flow strip. (f) The direct fluorescence stimulated by UV light was visualized to detect HCMV virus. The reaction was examined under UV light at 8, 10, 15 and 20 minutes after incubation at 37° C. (g) Twenty minutes after incubation, a lateral flow strip was dipped into the reaction tube for 5 minutes to visualize the control and test bands.

FIG. 6. Detection of SARS-CoV-2 using suboptimal PAM-mediated one-pot reaction. (a) Genomic map of canonical PAM (TTTV) and suboptimal PAM (VTTV, TTVV, TCTV) spacers in SARS-CoV-2. (b-e) Detection limits of FASTER (b, d) at 42° C. and STOPCovid.v1 (c, e) at 60° C. on DNA and RNA. The numbers of molecules input in FASTER and STOPCovid.v1 were the same. (f) FASTER results for 204 SARS-CoV-2 nasopharyngeal swab samples obtained from patients (left: 104 positive samples, 48 unextracted samples marked by solid circle and 56 pre-extracted samples marked by hollow circle; right: 100 negative samples). The fluorescence readout was measured at 20 minutes at 42° C. The threshold was determined as the three times of the average of all samples' initial fluorescence values, as S/N (signal-to-noise)=3. (g) Direct visualization under UV light to detect unextracted SARS-CoV-2 positive samples. The reaction was examined under UV light at 10, 15 and 20 minutes after incubation at 42° C. (h) Concordance table between FASTER and RT-qPCR for 204 samples.

FIG. 7. Suboptimal and canonical PAM-mediated one-pot detection. One-pot detection used spacers with suboptimal or canonical PAMs in Orflab (a) and E (b) genes of SARS-CoV-2. The crRNAs 1-3 targeting Orflab gene and crRNAs 2-7 targeting E gene used canonical PAMs whereas crRNAs 4-5 targeting Orflab gene and crRNA 1 targeting E gene used suboptimal PAMs. (c) The PAMs and spacers used in the one-pot reaction.

FIG. 8. Collateral activity and one-pot reaction comparison on various suboptimal and canonical PAMs. (a-d) Summary map of fluorescent kinetics for position 1-3 point-mutated suboptimal PAMs and three canonical PAMs in collateral activity test (a & b) and the corresponding one-pot reaction of HPV18 L1 gene spacer 1 (c) and SARS-CoV-2 S gene spacer 2 (d). Time to half-maximum fluorescence was determined. Fluorescence values were determined at 40 and 20 minute for collateral activities and one-pot reactions, respectively. 2.7 nM and 2.8 nM dsDNA substrates were added into collateral activity assays for HPV18 L1 gene spacer 1 and S gene spacer 2; 18.3 fM and 189 fM dsDNA substrates were added into one-pot reaction assays for HPV18 L1 gene spacer 1 and S gene spacer 2 which carried out at 37° C., n=3. The fluorescence detection of Orflab spacer 5 in collateral activity. The CTTA PAM was mutated to TTTA, TTTG and TTTC, and the T1-T3 in TTTV PAM were mutated to A, G and C respectively. The T1-T3 mutated PAM based on TTTA (a-c), TTTG (d-f) and TTTC PAM (g-i) were determined to compare the collateral activity.

FIG. 9. Collateral activities and one-pot reactions of Orflab spacer 4 using various PAMs. The GTTG PAM was mutated to TTTA, TTTG and TTTC as canonical PAMs and the T1-T3 in TTTV PAM were mutated to A, G or C respectively. (a-i) Collateral activities of the T1-T3 mutated suboptimal PAMs and relevant canonical PAMs were compared. (j-r) One-pot reactions of the T1-T3 mutated suboptimal PAMs and relevant canonical PAMs were compared. 3.5 nM dsDNA was added into collateral activity assays and 2.3 pM dsDNA was added into one-pot reaction assays which carried out at 37° C., n=3.

FIG. 10. Collateral activities and one-pot reactions of Orflab spacer 5 using various PAMs. The CTTA PAM was mutated to TTTA, TTTG and TTTC as canonical PAMs and the T1-T3 in TTTV PAM were mutated to A, G or C respectively. (a-i) Collateral activities of the T1-T3 mutated suboptimal PAMs and relevant canonical PAMs were compared. (j-r) One-pot reactions of the T1-T3 mutated suboptimal PAMs and relevant canonical PAMs were compared. 3.5 nM dsDNA was added into collateral activity assays and 2.3 pM dsDNA was added into one-pot reaction assays which carried out at 37° C., n=3.

FIG. 11. Collateral activities and one-pot reactions of HPV18 L1 gene spacer 1 using various PAMs. The TTAC PAM was mutated to TTTA, TTTG and TTTC as canonical PAMs and the T1-T3 in TTTV PAM were mutated to A, G or C respectively. (a-i) Collateral activities of the T1-T3 mutated suboptimal PAMs and relevant canonical PAMs were compared. (j-r) One-pot reactions of the T1-T3 mutated suboptimal PAMs and relevant canonical PAMs were compared. 2.7 nM dsDNA was added into collateral activity assays and 18.3 fM dsDNA was added into one-pot reaction assays which carried out at 37° C., n=3. The fluorescence detection of Orflab spacer 5 in one-pot reaction.

FIG. 12. Collateral activities and one-pot reactions of S gene spacer 2 using various PAMs. The TTCT PAM was mutated to TTTA, TTTG and TTTC as canonical PAMs and the T1-T3 in TTTV PAM were mutated to A, G or C respectively. (a-i) Collateral activities of the T1-T3 mutated suboptimal PAMs and relevant canonical PAMs were compared. (j-r) One-pot reactions of the T1-T3 mutated suboptimal PAMs and relevant canonical PAMs were compared. 2.8 nM dsDNA was added into collateral activity assays and 189 fM dsDNA was added into one-pot reaction assays which carried out at 37° C., n=3.

FIG. 13. Schematic of Cas12a recognizing and interacting with PAM-duplex. The A paired with the T2 directly forms hydrogen bonds with conserved Lys538 and Lys595 of Cas12a (modified from Yamano T, Mol Cell, 2017).

FIG. 14. The two-point and three-point mutated PAMs-mediated collateral activity and one-pot reaction. (a-d) The collateral activity (a, c) and one-pot reaction (b, d) targeting Orflab spacer 4 and 5 with TTNT PAMs. (e-f) The collateral activity (e) and one-pot reaction (f) targeting Orflab spacer 4 with VVTV, VTVV PAMs. (g-h) The collateral activity (g) and one-pot reaction (h) targeting Orflab spacer 5 with VVTV, VTVV and TCCV PAMs. (i j) The collateral activity (i) and one-pot reaction (j) targeting Orflab spacer 5 with CCCV and AGCV PAMs. 3.5 nM dsDNA was added into collateral activity assays and 2.3 pM dsDNA was added into one-pot reaction assays which carried out at 37° C., n=3.

FIG. 15. Comparison of suboptimal and canonical PAM-mediated one-pot reaction. E gene spacer 8 (a) and S gene spacer 3 (b) of SARS-CoV-2 were examined. The concentrations of dsDNA in one-pot reactions were 325.5 fM and 189 fM for E gene spacer 8 and S gene spacer 3, respectively. The one-pot reactions were carried out at 37° C.

FIG. 16. Determining the dose effect of RNP in the one-pot detection. RNP dose ranging from 5.5, 11, 22, 33, 66 to 132 nM were tested in the one-pot reaction with suboptimal PAM (a) and canonical PAM (b) at 37° C. The concentration of 2.3 pM dsDNA was added into one-pot reactions.

FIG. 17. Amount of RPA amplicons accumulated in one-pot reaction. Components of RPA, crRNA/Cas12a RNP and dsDNA substrate were incubated at 37° C. for 0, 2, 4, 6, 8, 10, 12, 14, 16, 18, 20 minutes and the RPA amplicons were analyzed in agarose gel. RPA alone represents one-pot reaction without crRNA/Cas12a RNP; TTTG, TTTC and TTCG, TTAC represent one-pot reaction with canonical PAM and suboptimal PAM, respectively. Arrows indicate the RPA amplicons.

FIG. 18. Cis-cleavage activities of 120 PAMs of four targets. In vitro cleavage activities of dsDNA substrates containing HPV18 L1 gene spacer 1, Orflab spacer 4, Orflab spacer 5 and S gene spacer 2 with suboptimal PAMs (VTTV, TVTV, TTVV) and canonical PAMs (TTTV). The RNP and dsDNA was incubated at 37° C. for 0, 1, 5, 10 or 20 minutes. The dsDNA substrates used for HPV18 L1 gene spacer 1, Orflab spacer 4, Orflab spacer 5 and S gene spacer 2 were 7.5 nM, 11 nM, 6 nM and 9 nM, respectively. HPV18 L1 gene spacer 1, S: 591 bp, P: 382 bp, 209 bp; Orflab spacer 4, S: 539 bp, P: 388 bp, 151 bp; Orflab spacer 5, S: 461 bp, P: 220 bp, 241 bp; S gene spacer 2, S: 570 bp, P: 257 bp, 313 bp.

FIG. 19. Comparison of AMP future- and TwistDx-based one-pot reaction. (a) Amplification comparison using AMP future and TwistDx kit. The RPA amplification was performed at 42° C. for 20 minutes. (b) Fluorescence comparison of AMP future- and TwistDx-based one-pot reaction. 8403 aM, 840.3 aM, 84.03 aM, 8.403 aM dsDNA were applied. (c) RNP dose optimization in TwistDx-based one-pot reaction. 333, 200, 100 or 33.3 nM RNP were applied. 8403 aM DNA substrate was used. Suboptimal PAM was used in b-c. (d-g) Comparison of suboptimal and canonical PAM mediated one-pot reaction. Suboptimal PAM and canonical PAM for Orflab spacer4, Orflab spacer 5, E gene spacer 8 of SARS-CoV-2 and L1 gene spacer 1 of HPV18 were compared using TwistDx kit and 100 nM RNP. The concentrations of dsDNA substrates input were 2340 fM for Orflab spacer 4 and spacer 5, 325.5 fM for E gene spacer 8 and 243.9 fM for HPV18 L1 gene spacer1, respectively.

FIG. 20. The numbers of spacers with canonical and suboptimal PAM counted in HCMV and SARS-CoV-2. (a) Spacers with canonical (TTTA, TTTG, TTTC) and suboptimal PAMs (VTTV, TTVV, TCTV) in HCMV. (b-c) Canonical and Alternative suboptimal PAMs (A-suboptimal PAM, TTNT, TRTV, YYYN (except TTTV)) in SARS-CoV-2 (b) and HCMV (c).

FIG. 21. Optimization of reverse transcription reaction. (a) One-pot reaction was carried out at 42° C., 43° C. and 45° C. (b) High-efficiency primers screen for reverse transcription enzyme. RT products were quantified by qPCR after RT-enzyme reacting at 48° C. for 30 mins. (c-d) UV images and fluorescence of FASTER with variable concentration of RPA reverse primer (RPA-R) and reverse transcription primer 1 (RT-1) using extracted viral samples. NC represents reactions without substrates.

FIG. 22. Limit of detection (LOD) of RT-qPCR on SARS-CoV-2 virus-like particles. (a) LOD of RT-qPCR using CDC N2 primer pairs. The input substrates were 10⁴, 10³, 10², 10¹, 10^{0.5}, 10⁰ and 10^{-0.5} copies/μL. (b) CDC RT-qPCR assay standard curve. Standard curve generated by tenfold dilutions of the input substrates, with three replicates for each dilution. (c) Detection limits of FASTER on SARS-CoV-2 virus-like particles.

FIG. 23. FASTER on patient samples. (a-b) positive NP swabs detected by UV light imaging. (a) unextracted samples and (b) extracted samples. UV images were captured at 15-20 min. (c) Unextracted NP swabs with varying Ct values imaged by UV light and simple blue light device. (left: 0 min, right: 20 min)

FIG. 24. STOPCovid, version1 (STOPCovid.v1) on patient samples. (a-b) 104 positive patient samples and 19 negative patient samples were detected by STOPCovid.v1. 48 unextracted samples were marked by solid circle and 56 extracted samples were marked by hollow circle, respectively. The fluorescence values were read at 45 min. (c) The results evaluation of STOPCovid.v1 and RT-qPCR.

FIG. 25. Specificity evaluation of FASTER. (a) Alignment of SARS-CoV-2 N gene interest region with common human coronaviruses N gene, including MERS, HKU1, 229E, NL63 and OC43; (b) Identification of amplicons by RPA assays with 1E5 copies per reaction of each coronaviruses N gene dsDNA at 37° C. for 15 minutes; Arrow represents amplicons. (c) Fluorescence kinetics of collateral activity tested with 2.5 nM dsDNA input of different sequences at 37° C.; (d) Fluorescence kinetics of one-pot reaction tested with 1E5 copies per reaction of each coronaviruses N gene RNA at 42° C.

FIG. 26. Comparison of CRISPR-based SARS-CoV-2 detection methods. The substrates for evaluating sensitivity are the following: SARS-CoV-2 virus-like particles for FASTER, N gene RNA for DETECTR and Amplification-free detection, extracted genomic RNA for SHERLOCK and SHINE, SARS-CoV-2 genome standards for STOPCovid.v1, and concentrated samples for STOPCovid.v2.

FIG. 27. The LbCas12a mutants mediated faster one-pot reaction than the Wild-type protein. (a-g) The PAM-relevant residues 595K and 595K&542Y of LbCas12a protein were mutated to alanine. One-pot reaction of LbCas12a WT and mutants on Orflab gene (a-b), E gene (c-d) and N gene (e-g). The reaction were carried out at 42° C. with 20 pg dsDNA substrate for a-d, and various doses for e-g.

FIG. 28. The AapCas12b mutants mediated faster one-pot reaction than the Wild-type protein. (a) Cis-cleavage of AapCas12b WT and 3M on 100 ng N gene dsDNA. NC means reaction without protein. S is substrate and P1 and P2 are cleaved products. (b) Trans-cleavage activity of WT and 3M. (c) one-pot reaction combined RPA and AapCas12b. Reactions were carried out at 42° C. (d-e) StopCovid.v1 reaction of WT and 3M at 60° C. (f) Fluorescence values of StopCovid.v1 reaction at 25 min. 3M mutant referred to G478A/K396A/Q403A of AapCas12b.

### DETAILED DESCRIPTION

### One-Step Detection of Nucleic Acids

A one-step, fast, sensitive, reliable and flexible method for detecting nucleic acids is provided, in one embodiment of the present disclosure. This method showed comparable detection limit to quantitative PCR (qPCR) but with significant shorter time, e.g., from 15 to 20 minutes.

It was discovered that, when the reagents for both isothermal amplification and CRISPR detection are pooled to enable one-step detection, the CRISPR machinery can prematurely cleave the substrate nucleic acid which serves as template for amplification. This results in insufficient or slow amplification, which causes missed detection or inefficient detection. Systems and methods are provided, in some embodiments of the present disclosure, to orchestrate the amplification and CRISPR detection processes, so that the amplification is can be efficiently carried out, allowing fast and efficient detection of the amplified products.

In accordance with one embodiment of the present disclosure, therefore, provided is a method for detecting a target polynucleotide, comprising incubating the target polynucleotide in a mixture that comprises (a) a polymerase, (b) deoxynucleoside triphosphates (dNTPs), (c) primers for amplifying the target polynucleotide, (c) a CRISPR-associated (Cas) nuclease, and (d) a guide RNA comprising a spacer fragment complementary to a target fragment on the target polynucleotide.

In some embodiments, the polymerase, the primers, and the dNTPs are able to amplify the target polynucleotide isothermally. Isothermal amplification techniques are well known in the art. Isothermal amplification methods provide detection of a nucleic acid target sequence in a streamlined, exponential manner, and are not limited by the constraint of thermal cycling. Although these methods can vary considerably, they all share some features in common. For example, because the DNA strands are not heat denatured, all isothermal methods rely on an alternative approach to enable primer binding and initiation of the amplification reaction: a polymerase with strand-displacement activity. Once the reaction is initiated, the polymerase must also separate the strand that is still annealed to the sequence of interest.

Isothermal methods typically employ unique DNA polymerases for separating duplex DNA. DNA polymerases with this ability include Klenow exo-, Bsu large fragment, and EquiPhi29, phi29 for moderate temperature reactions (25-40° C.) and the large fragment of Bst, Bsm DNA polymerase for higher temperature (50-65° C.) reactions. To detect RNA species, a reverse transcriptase compatible with the temperature of the reaction is added (except in the NASBA/TMA reaction) to maintain the isothermal nature of the amplification.

One example of isothermal amplification is Loop-Mediated Isothermal Amplification (LAMP). LAMP uses 4-6 primers recognizing 6-8 distinct regions of target DNA. A strand-displacing DNA polymerase initiates synthesis and 2 of the primers form loop structures to facilitate subsequent rounds of amplification. LAMP is rapid, sensitive, and amplification is so extensive that the magnesium pyrophosphate produced during the reaction can be seen by eye, making LAMP well-suited for field diagnostics.

Another example of isothermal amplification is Whole Genome Amplification (WGA). WGA is a method of Multiple Displacement Amplification (MDA) that utilizes the strand-displacement activity of DNA polymerases such as EquiPhi29, phi29 or Bst, Bsm DNA Polymerase to enable robust amplification of an entire genome. WGA has become an invaluable approach for utilizing limited samples of precious stock material or to enable sequencing of single-cell genomic DNA. Products of the reaction are extremely long (>30 kb) and highly branched through the multiple displacement mechanism.

Another example of isothermal amplification is Strand Displacement Amplification (SDA). SDA, or a similar approach, Nicking Enzyme Amplification Reaction (NEAR), relies on a strand-displacing DNA polymerase, typically Bst DNA Polymerase, Large Fragment or Klenow Fragment (3′-5′ exo-), to initiate at nicks created by a strand-limited restriction endonuclease or nicking enzyme at a site contained in a primer. The nicking site is regenerated with each polymerase displacement step, resulting in exponential amplification. NEAR is extremely rapid and sensitive, enabling detection of small target amounts in minutes.

Another example of isothermal amplification is Helicase-Dependent Amplification (HDA). HDA employs the double-stranded DNA unwinding activity of a helicase to separate strands, enabling primer annealing and extension by a strand-displacing DNA polymerase. Like PCR, this system requires only two primers.

Another example of isothermal amplification is Recombinase Polymerase Amplification (RPA). RPA uses a recombinase enzyme to help primers invade double-stranded DNA. T4 UvsX, UvsY, and a single stranded binding protein T4 gp32 form D-loop recombination structures that initiate amplification by a strand-displacing DNA polymerase. RPA is typically performed at −37-42° C. and, unlike other methods, can produce discrete amplicons up to 1 kb.

Another example of isothermal amplification is Nucleic Acid Sequences Based Amplification (NASBA). NASBA and Transcription Mediated Amplification (TMA) are both isothermal amplification methods that proceed through RNA. Primers are designed to target a region of interest; one of the primers must include the promoter sequence for T7 RNA polymerase at the 5′ end. And there are other isothermal amplification including rolling circle amplification (RCA), asymmetric isothermal amplification (SMAP 2), Exponential Amplification Reaction (EXPAR), Beacon-Assisted Detection Amplification, Single primer isothermal amplification (SPIA), cross priming amplification (CPA).

In some embodiments, the ingredient(s) or conditions are tuned such that the polymerase can effectively amplify the target polynucleotide while the Cas nuclease is capable of cleaving the amplified target polynucleotide. In one embodiment, at least 10², 10³, 10⁴, 10⁵, 10⁶, 10⁷, 10⁸, 10⁹, 10¹⁰, 10¹¹, 10¹², 10¹³, or 10¹⁴ times of amplification of the target polynucleotides is achieved within 10 minutes, while the Cas nuclease and the guide RNA are present in the mixture.

This can be achieved by multiple means. In one example, the binding between the Cas nuclease (or, Cas protein) and the guide RNA, or between the Cas-guide RNA ribonucleoprotein (RNP) and the target polynucleotide, is reduced. In another example, the cleavage efficiency of the Cas protein is reduced.

A. Suboptimal PAM

An example method of reducing the binding between the RNP and the target polynucleotide is to design the guide RNA to target a suboptimal PAM. A protospacer adjacent motif (PAM) is a 2-8-base pair DNA sequence immediately following the sequence targeted by a Cas nuclease in the CRISPR bacterial adaptive immune system. PAM is an essential targeting component. Each Cas nuclease has one or more canonical PAM sequences, as well as some non-canonical ones. The non-canonical PAMs are not optimal and can lead less efficient binding and cleavage.

In one embodiment, the guide RNA is designed so that it includes, or is adjacent to, a protospacer adjacent motif (PAM) sequence recognizable by the Cas nuclease, and is suboptimal or non-canonical.

For each known Cas nuclease, the corresponding canonical and non-canonical PAMs are known. For instance, for LbCas12a, the non-canonical PAM sequences include NTTV, TNTV, TTNV (except TTTV), TTNT, VTTT, TVTT, VVTT, VTVT, VNVV, NVNV, NVVV, VNTV, NTVV, TNVV, and VVNV, YYYN wherein N denotes any nucleotide. For AapCas12b, the non-canonical PAM sequences VTN, TTN (except TTV), TVN, NVN and VVN, wherein N denotes any nucleotide.

B. Modified Cleavage Substrate

Certain modifications to the target polynucleotide for a CRISPR system may reduce the binding affinity and/or cleavage efficiency, while not impacting the amplification. Therefore, by incorporating these modifications to the substrate, the Cas cleavage can be inhibited or deferred to allow sufficient amplification.

Such modifications can be incorporated into the amplified target polynucleotide through modified dNTP, and/or modified primers.

In some embodiments, the dNTP is substituted with an analog or variant, such as deoxyuridine triphosphate, a deoxyinosine triphosphate, a pseudouridin triphosphate, a methylpseudouridin triphosphate, 2-aminopurine, 5-bromo dU or a ribonucleoside triphosphate (rNTP). In some embodiments, the dNTP, the rNTP, or any of the analogs or variant may be modified. Non-limiting examples of modifications include those with a group such as phosphoryl, biotin, digoxigenin, amino, thiol, phosphorthioate, and methyl.

In some embodiments, one or more of the nucleotides in the primer(s) is substituted with an analog or variant, such as deoxyuridine triphosphate, a deoxyinosine triphosphate, a pseudouridin triphosphate, a methylpseudouridin triphosphate, 2-aminopurine, 5-bromo dU or a ribonucleoside triphosphate (rNTP). In some embodiments, the nucleotide, the rNTP, or any of the analogs or variant may be modified. Non-limiting examples of modifications include those with a group such as phosphoryl, biotin, digoxigenin, amino, thiol, phosphorthioate, and methyl.

The percentage of such modified nucleotides can be adjusted based on needs. Higher percentages of modifications can reduce the CRISPR binding/cleavage efficiency more, and vice versa. In some embodiments, at least 1%, 2%, 3%, 4%, 5%, 6%, 7%, 8%, 9%, 10%, 15% or 20% of the dNTP, or nucleotides within the primers, are modified. In some embodiments, no more than 10%, 11%, 12%, 13%, 14%, 15%, 20%, 25%, 30%, 35%, 40%, 45% or 50% or 60% or 70% of the dNTP, or nucleotides within the primers, are modified.

Primers of the reaction can be chemically modified to improve the isothermal amplification or, in some embodiments, primers have partial sequence of the crRNA spacers or have partial or all sub-optimal PAM sequences, and primers can be chemically modified or not modified.

C. Modified Guide RNA

In another example, the guide RNA (or the crRNA) is modified, as compared to standard guide RNA structures, to inhibit the formation of the RNP or the binding between the RNP and the target nucleotide. Examples of modified guide RNA/crRNA are provided below.

In some embodiments, the crRNA is truncated in the 3′ end of the guide region. The truncated crRNA, in some embodiments, contains 10, 11, 12, 13, 14, 15, 16, 17, 18, 19 or even less complementary nucleotides to the target polynucleotide.

In some embodiments, the crRNA is truncated at the 5′ end of hairpin region. In some embodiments, the guide RNA includes a truncated tracrRNA sequence. In some embodiments, the truncation is with 1, 2, 3, 4 or 5 nucleotides.

In some embodiments, the hairpin structure of the crRNA is extended, e.g., at the 3′ end of the spacer (e.g., 5′-AGACAUGGACCA-3′). In some embodiments, the stem region includes an extended sequence. In some embodiments, the loop region includes an extended sequence. The extension is for at least 1, 2, 3, 4, 5, 10, 15, 20, 25 or 30 nucleotides. The length of the hairpin sequence could be 1 to 100 nt or even longer.

In some embodiments, the crRNA or tracrRNA is extended at the 5′ end and(or) 3′ end, e.g., at the 3′ end of the spacer (e.g., 5′-AGACAUGGACCA-3′). The extension is for at least 1, 2, 3, 4, 5, 10, 15, 20, 25 or 30 nucleotides. The length of the sequence may be 1 to 100 nt or even longer, meanwhile, any modification expected can be incorporated.

In some embodiments, the nucleotides of the guide RNA which interact with Cas protein, through the 2′ hydroxyl group of ribose, are replaced by DNA. In some embodiments, the nucleotides (1-12nt, longer or entire sequence) of the 5′ end of the spacer, either continuous or discontinuous, can be modified.

In some embodiments, one or more nucleotides in the guide regions of the guide RNA incorporate one or more locked nucleic acids (LNAs) or bridged nucleic acids (BNAs). In some embodiments, the one or more nucleotides are at positions of 1-12nt or 12-20nt within the guide region.

Other example modifications to the nucleotides in the guide RNA includes deoxynucleotide, a locked nucleic acid (LNA), a bridged nucleic acid (BNA), a deoxyuridine, a deoxyinosine, a pseudouridin, a methylpseudouridin, or modified nucleotides with a group such as phosphoryl, biotin, digoxigenin, amino, thiol, phosphorthioate, methyl, 2′-O-methyl-3′-phosphonoacetate (MP), 2′O-methoxyethyl (MOE), Fluoro(F), S-constrained ethyl, 2′-O-methyl-PS (MS) and 2′-O-methyl-thioPACE (MSP).

In some embodiments, the guide RNA includes 1, 2, 3, 4, or 5 or 6 mismatches in the complimentary region to the target polynucleotide (the spacer). The mismatch may be consecutive or discontinuous.

D. Cas Nuclease

In another embodiment, an engineered Cas nuclease is employed that has reduced binding to the guide RNA and/or the target polynucleotide, or reduced cleavage activity.

The term “Cas nuclease,” “Cas protein,” or “clustered regularly interspaced short palindromic repeats (CRISPR)-associated (Cas) protein” refers to RNA-guided DNA or RNA endonuclease enzymes associated with the CRISPR (Clustered Regularly Interspaced Short Palindromic Repeats) adaptive immunity system in Streptococcus pyogenes, as well as other bacteria. Non-limiting examples of Cas proteins include Streptococcus pyogenes Cas9 (SpCas9), Staphylococcus aureus Cas9 (SaCas9), Acidaminococcus sp. Cas12a (AsCpf1), Lachnospiraceae bacterium Cas12a (LbCpf1), Francisella novicida Cas12a (FnCpf1). Additional examples are provided in Komor et al., “CRISPR-Based Technologies for the Manipulation of Eukaryotic Genomes,” Cell. 2017 Jan. 12; 168(1-2):20-36.

Non-limiting examples of Cas nucleases include Cas12a, Cas12b, Cas12c, Cas12d, Cas12e (CasX), Cas 12f, Cas12k, Cas13a, Cas13b, Cas13c, Cas13d, Csm6, Csm3 and Cas14a, Cas14b, and Cas14c. More specific examples include AsCas12a, FnCas12a, MbCas12a, Lb3Cas12a, Lb2Cas12a, BpCas12a, PeCas12a, PbCas12a, SsCas12a, CMtCas12a, EeCas12a, LiCas12a, PcCas12a, PdCas12a, PmCas12a, ArCas12a, HkCas12a, ErCas12a, BsCas12a, LpCas12a, PrCas12a, and PxCas12a (of class Cas12a), AapCas12b, AmCas12b, AacCas12b, BsCas12b, BvCas12b, BthCas12b, BhCas12b, AkCas12b, EbCas12b, LsCas12b (of class Cas12b), Mi1Cas12f2, Mi2Cas12f2, Un1Cas12f1, Un2Cas12f1, AuCas12f2, PtCas12f1, AsCas12f1, RuCas12f1, SpCas12f1, and CnCas12f1 (of class Cas12f (Cas14a)), ShCas12k (CAST), and AcCas12k (of class Cas12k), LwaCas13a, LbaCas13a, LshCas13a, PprCas13a, EreCas13a, LneCa3a, CamCas13a, RcaCas13a, HheCas13a, LbuCas13a, LseCas13a, LbmCas13a, LbnCas13a, RcsCas13a, RcrCas13a, RcdCas13a, CgCas13a, Cg2Cas13a, LweCas13a, LbfCas13a, Lba4Cas13a, Lba9Cas13a, LneCas13a, HheCas13a, and RcaCas13a (of class Cas13a), BzCas13b, PbCas13b, PspCas13b, RanCas13b, PguCas13b, PsmCas13b, CcaCas13b, AspCas13b, PauCas13b, Pin2Cas13b, and Pin3Cas13b (of class Cas13b), RspCas13d, RfxCas13d, EsCas13d, and AdmCas13d (of class Cas13d), and TtCsm6, EiCsm6, and LsCsm6 (of class Csm6).

Example mutations to reduce the binding to the guide RNA and/or the target polynucleotide, or reduce the cleavage activity of the Cas nuclease are provided in the tables below.

Based on structural analysis, the residues in Table A relate to the formation of ribonucleoprotein (RNP).

TABLE A
Residues Impacting RNP formation
Residues related to RNP formation of LbCas12a

| | | | | | | | |
|---|---|---|---|---|---|---|---|
| Arg386 | Lys390 | Gln264 | Lys253 | Lys464 | Arg508 | Arg174 | Lys278 |
| Leu281 | Arg158 | Asn157 | Lys51 | Lys520 | Arg18 | His720 | Thr16 |
| Lys15 | Asn718 | Lys953 | Lys960 | Thr716 | Thr713 | His714 | Gly715 |
| Ser710 | His714 | Asn808 | Ala766 | Asn767 | Thr777 | Lys774 | Asp786 |
| Tyr781 | Arg788 | Asn772 | Asn769 | Lys768 | Lys707 | Tyr872 | Glu898 |
| Gln793 | Arg747 | Lys20 | | | | | |

Residues related to RNP formation of AapCas12b

| | | | | | | | |
|---|---|---|---|---|---|---|---|
| Leu978 | Gln982 | Gln973 | Arg415 | Met443 | His614 | Phe600 | Tyr839 |
| Arg815 | Glu819 | Gln618 | Arg746 | Ala794 | Arg792 | Arg738 | Arg731 |
| Arg734 | Thr796 | Val753 | Gly754 | Gln767 | Leu764 | Arg738 | Val737 |
| His800 | Pro743 | Asn766 | Asp807 | His803 | Arg484 | Tyr501 | Lys9 |
| Ser442 | Arg742 | Lys744 | Gly755 | Ile745 | Arg746 | Gln446 | Lys810 |
| Ser5 | Asn503 | Asn881 | Lys7 | Trp391 | Arg485 | Tyr825 | Lys811 |
| Trp835 | Gln882 | Asp814 | Lys4 | | | | |

Residues related to RNP formation of LbuCas13a

| | | | | | | | |
|---|---|---|---|---|---|---|---|
| Asn139 | Tyr245 | Asn142 | Lys237 | Gln271 | His294 | Arg233 | Glu297 |
| Ser301 | His228 | Arg224 | Tyr276 | Lys275 | Ser143 | Ser147 | Tyr274 |
| Asn151 | Arg172 | Tyr176 | Lys340 | Asn339 | Lys336 | Tyr307 | Arg311 |
| Lys1087 | Phe1102 | Lys319 | Arg322 | Ala1106 | Arg1072 | Asn1083 | Ser1088 |
| Ser780 | Phe375 | Lys783 | Asn378 | Arg963 | Arg973 | Lys5 | His11 |
| Ser363 | His962 | His11 | Ala367 | Ala787 | Glu371 | Ser10 | Thr4 |
| Val3 | Lys2 | Lys305 | Met1 | Lys140 | Lys2 | Ser555 | Lys558 |
| Tyr601 | Asn549 | Asn547 | Asn804 | Arg809 | His771 | Arg547 | Asn804 |
| Arg857 | Lys942 | Tyr938 | His901 | | | | |

Residues related to RNP formation of AsCas12a

| | | | | | | | |
|---|---|---|---|---|---|---|---|
| Lys414 | Gln286 | Lys273 | Lys369 | Gly270 | His479 | Asn515 | His479 |
| Arg518 | Gln956 | Trp382 | Arg192 | Lys307 | Leu310 | Arg176 | Lys51 |
| Asn175 | Tyr47 | Glu786 | His872 | Lys530 | His761 | Thr16 | Lys15 |
| Lys1022 | His977 | Lys1029 | Lys757 | His856 | Leu807 | Met806 | Asn808 |
| Lys852 | Ile858 | Lys810 | Lys809 | Arg863 | Lys943 | Tyr940 | Asp966 |
| Arg790 | Lys748 | Arg18 | Asn759 | Lys752 | Ser973 | Leu760 | Arg313 |
| Arg955 | | | | | | | |

Residues related to RNP formation of AacCas12b

| | | | | | | | |
|---|---|---|---|---|---|---|---|
| Leu978 | Gln982 | Gln973 | His514 | Phe600 | Tyr839 | Arg815 | Glu819 |
| Gln618 | Arg415 | Met443 | Ala794 | Arg792 | Arg738 | Arg731 | Arg734 |
| Thr796 | Arg746 | Leu764 | Arg738 | Gln767 | Val737 | His800 | Pro743 |
| Val753 | Gly754 | Asn756 | Asp807 | His803 | Arg484 | Tyr501 | Lys9 |
| Ser442 | Arg742 | Lys744 | Ile745 | Gly755 | Arg746 | Gln446 | Lys810 |
| Ser5 | Asn503 | Asn881 | Lys7 | Trp391 | Arg485 | Tyr825 | Lys811 |
| Trp835 | Gln882 | Asp814 | Lys4 | | | | |

The residues in Table B are contemplated to be important to maintain the conformation of the Cas nucleases.

TABLE B
Residues Important for Cas Conformation
Residues undergoing conformational change of LbCas12a

| | | | | |
|---|---|---|---|---|
| Val511 | Thr512 | Try890 | Gln888 | Lys457 |

Residues undergoing conformational change of LbuCas13a

| | | | | |
|---|---|---|---|---|
| His962 | Lys783 | Gln371 | Phe375 | Lys5 |
| Lys2 | | | | |

The residues in Table C are contemplated to be involved in the interaction with the PAM sequence.

TABLE C
Residues interacting with PAM
Residues interacting with PAM of LbCas12a

| | | | | |
|---|---|---|---|---|
| Ser599 | Lys600 | Try646 | Lys601 | Lys538 |
| Trp649 | Tyr542 | Asp542 | Asp535 | Trp534 |
| Gly740 | Thr148 | Thr149 | Lys121 | Lys595 |
| Try616 | | | | |

Residues interacting with PAM of AapCas12b

| | | | | |
|---|---|---|---|---|
| Lys209 | Lys141 | Ala142 | Lys145 | Asn144 |
| Gly143 | Asn400 | Lys396 | Gln118 | Gly478 |
| Arg507 | Gln403 | Arg208 | Gln211 | Ala212 |
| Val213 | Arg218 | Val134 | Gly135 | Leu137 |
| Gly136 | Gln119 | Arg122 | Gly143 | Asn144 |

Residues interacting with PAM of AsCas12a

| | | | | |
|---|---|---|---|---|
| Lys164 | Thr166 | Thr167 | Ala135 | Lys607 |
| Lys613 | Asn631 | Tyr687 | Asn547 | Lys689 |
| Asp545 | Lys548 | Lys780 | Asn782 | Gly783 |

Residues interacting with PAM of AacCas12b

| | | | | |
|---|---|---|---|---|
| Lys209 | Lys141 | Ala142 | Lys145 | Asn144 |
| Gly143 | Asn400 | Lys396 | Gln118 | Gly478 |
| Arg507 | Gln403 | Arg208 | Gln211 | Ala212 |
| Val213 | Arg218 | Val134 | Gly135 | Leu137 |
| Gly136 | Gln119 | Arg122 | Gly143 | Asn144 |
| Arg150 | Arg147 | | | |

Conserved amino acids in the REC1 (24-282) and REC2 (283-521) domains and the Helical-I (14-391) and Helical-II (660-822) domains are contemplated to be important to the structure or activity of the Cas nucleases. Another example is residues in the HNH domain.

In addition, residues on the REC lobe, Nuc lobe, or RuvC domain that form hydrogen bonds with target DNA can also be the target for mutations. Additional suitable targets for mutations are the positively charged residues or negatively charged ones. Examples are provided in Table D below.

TABLE D
Residues Interacting with Target DNA or RNA
Residues forming hydrogen bonds with target DNA of LbCas12a

| | | | | | | | |
|---|---|---|---|---|---|---|---|
| Asn260 | Asn256 | Ser288 | Ser286 | Lys514 | Ser168 | Lys167 | Asn160 |
| Trp355 | Lys897 | Ile896 | Lys945 | Gln944 | Phe983 | Lys984 | Gly740 |

Residues forming hydrogen bonds with target DNA of AapCas12b

| | | | | | | | |
|---|---|---|---|---|---|---|---|
| Arg208 | Gln403 | Gln211 | Ala212 | Val213 | Arg218 | Lys145 | Ala142 |
| Lys141 | Lys209 | Ser5 | Ser505 | Gln222 | Ala117 | Gln109 | Ser233 |
| Arg237 | Trp234 | Arg873 | Gly874 | Phe855 | Lys805 | Arg798 | Phe793 |
| Arg785 | Lys789 | Lys284 | Glu285 | His289 | Leu281 | Thr292 | Ser335 |
| Arg294 | Arg297 | Ser862 | Pro86 | Gln866 | Arg331 | Gly955 | Trp930 |
| Arg332 | Arg900 | Phe897 | Ser898 | Ser899 | Asp570 | Glu848 | Asp977 |
| Arg911 | Leu573 | Arg574 | Arg913 | Asn1101 | Gln1093 | Arg859 | Tyr853 |

Residues forming hydrogen bonds with target RNA of LbuCas13a

| | | | | | | | |
|---|---|---|---|---|---|---|---|
| Lys47 | Arg41 | Lys86 | Gln659 | Arg1135 | Gln904 | Thr557 | Asp590 |
| Lys597 | Ser522 | Gln519 | Arg809 | Val810 | Arg857 | Lys855 | Asn997 |
| Glu996 | Lys998 | His477 | Gln1007 | His473 | Phe995 | Lys2 | |

Residues forming hydrogen bonds with target DNA of AsCas12a

| | | | | | | | |
|---|---|---|---|---|---|---|---|
| Ser376 | Asn282 | Asn278 | Arg301 | Thr315 | Lys524 | Arg955 | Arg951 |
| Ile964 | Lys965 | Gln1014 | Phe1052 | Ala1053 | Ser186 | Asn178 | Lys603 |
| Gln784 | Lys780 | Gly783 | Ser1051 | Gln1013 | Lys968 | Gly263 | Trp382 |

Residues forming hydrogen bonds with target DNA of FnCas12a

| | | | | | | | |
|---|---|---|---|---|---|---|---|
| Lys1066 | Lys895 | Lys1281 | Lys1287 | Lys1069 | Arg1016 | Phe1010 | Arg1014 |
| Asn1288 | Leu329 | Asp331 | Ser330 | Gln327 | Lys326 | Leu324 | Leu1008 |
| Gln1006 | Asp917 | Asn1009 | Arg1014 | Lys1021 | Arg1016 | Lys1013 | Gln309 |
| Asn305 | Leu306 | Glu302 | Asn301 | | | | |

Residues forming hydrogen bonds with target DNA of AacCas12b

| | | | | | | | |
|---|---|---|---|---|---|---|---|
| Arg332 | Arg900 | Phe897 | Ser898 | Ser899 | Asp570 | Glu848 | Asp977 |
| Arg911 | Leu573 | Arg574 | Arg913 | Asn1101 | Gln1093 | Arg859 | Tyr853 |
| Gln866 | Arg331 | Gly955 | Trp930 | Arg294 | Arg297 | Ser862 | Pro86 |
| Arg208 | Gln403 | Gln211 | Ala212 | Val213 | Arg218 | Lys145 | Ala142 |
| Lys141 | Lys209 | Ser5 | Ser505 | Gln222 | Ala117 | Gln109 | Ser233 |
| Arg237 | Trp234 | Arg873 | Gly874 | Phe855 | Lys805 | Arg798 | Phe793 |
| Arg785 | Lys789 | Lys284 | Glu285 | His289 | Leu281 | Thr292 | Ser335 |

The above identified amino acid residues, in some embodiments, can be deleted to substituted with a different amino acid. In some embodiments, the substitution is non-conservative substitution.

Whether a substitution is a non-conservative substitution can be determined with commonly known knowledge, such as with the matrix in Table E below. In Table E, a negative similarity score indicates non-conservative substitution between the two amino acids.

TABLE E
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, the substitution is with alanine.

E. Adjusted Reaction Conditions

In another embodiment, the reaction conditions are adjusted to favor amplification over CRISPR cleavage.

In one example, the amount of the Cas nuclease/guide RNA in the mixture is adjusted to reduce the cleavage efficiency. In another example, the magnesium ion concentration is increased or decreased to reduce the cleavage efficiency or increase amplification efficiency.

In another example, a certain amount of dimethyl sulfoxide (DMSO), bovine albumin (BSA), tween20, proteinase K inhibitor, or a nuclease inhibitor is added to the reaction system. In another example, the pH is adjusted, e.g., between 5.0 and 10.0, for the reaction mixture. In another example, the reaction temperature is adjusted.

In yet another example, one more additives (see examples in Table F) is added to the reaction mixture to reduce binding between the RNP and the target polynucleotide.

TABLE F
Example Additives

| | |
|---|---|
| Barium chloride dihydrate | Ethylenediaminetetraacetic acid |
| | disodium salt dihydrate |
| Cadmium chloride hydrate | Polyvinylpyrrolidone K15 |
| Calcium chloride dihydrate | Dextran sulfate sodium salt (Mr 5,000) |
| Cobalt(II) chloride hexahydrate | Pentaerythritol ethoxylate (3/4 EO/OH) |
| Copper(II) chloride dihydrate | Polyethylene glycol 3,350 |
| Magnesium chloride hexahydrate | D-(+)-Glucose monohydrate |
| Manganese(II) chloride tetrahydrate | Sucrose |
| Strontium chloride hexahydrate | Xylitol |
| Yttrium(III) chloride hexahydrate | D-Sorbitol |
| Zinc chloride | myo-Inositol |
| Iron(III) chloride hexahydrate | D-(+)-Trehalose dihydrate |
| Nickel(II) chloride hexahydrate | D-(+)-Galactose |
| Chromium(III) chloride hexahydrate | Ethylene glycol |
| Praseodymium(III) acetate hydrate | Glycerol |
| Ammonium sulfate | NDSB-195 |
| Potassium chloride | NDSB-201 |
| Lithium chloride | NDSB-211 |
| Sodium chloride | NDSB-221 |
| Sodium fluoride | NDSB-256 |
| Sodium iodide | CYMAL ® -7 |
| Sodium thiocyanate | Benzamidine hydrochloride |
| Potassium sodium tartrate | n-dodecyl-N,N-dimethylamine-N-oxide, |
| tetrahydrate | (LDAO, DDAO) |
| Sodium citrate tribasic dihydrate | n-Octyl-b-D-glucoside |
| Cesium chloride | n-Dodecyl-b-D-maltoside |
| Sodium malonate pH 7.0 | Trimethylamine N-oxide dihydrate |
| L-Proline | 1,6-Hexanediol |
| Phenol | (+/−)-2-Methyl-2,4-pentanediol |
| Dimethyl sulfoxide | Polyethylene glycol 400 |
| Sodium bromide | Jeffamine ® M-600 ® pH 7.0 |
| 6-Aminohexanoic acid | 2,5-Hexanediol |
| 1,5-Diaminopentane dihydrochloride | (±)-1,3-Butanediol |
| 1,6-Diaminohexane | Polypropylene glycol P 400 |
| 1,8-Diaminooctane | 1,4-Dioxane |
| Glycine | Ethanol |
| Glycyl-glycyl-glycine | 2-Propanol |
| Taurine | Methanol |
| Betaine hydrochloride | 1,2-Butanediol |
| Spermidine | tert-Butanol |
| Spermine tetrahydrochloride | 1,3-Propanediol |
| Hexammine cobalt(III) chloride | Acetonitrile |
| Sarcosine | Formamide |
| Trimethylamine hydrochloride | 1-Propanol |
| Guanidine hydrochloride | Ethyl acetate |
| Urea | Acetone |
| b-Nicotinamide adenine dinucleotide hydrate | Dichloromethane |
| Adenosine-5′-triphosphate | 1-Butanol |
| disodium salt hydrate | |
| TCEP hydrochloride | 2,2,2-Trifluoroethanol |
| GSH (L-Glutathione reduced), | 1,1,1,3,3,3-Hexafluoro-2-propanol |

With any one or a combination of these approaches, the target polynucleotide can be amplified sufficiently, followed by the guide RNA-guided cleavage by the Cas nuclease. The cleaved target polynucleotide can then be detected with a variety of different technologies known in the art.

For instance, the cleaving event may be detected with a toehold switch sensor, which can generate colorimetric output on test paper. Toehold switches are synthetic RNAs that mimic messenger RNAs whose job it is to shuttle information from the DNA to the protein-synthesizing machinery. They contain a recognition sequence (toehold) for a specific stimulus in form of a specific “input” RNA, and a recognition sequence that the protein-synthesizing machinery (ribosome) needs to bind to initiate the translation of a fused protein-coding sequence into its encoded protein product. In the absence of the “input” RNA, the toehold switch is kept in its OFF state by forming a hairpin structure that uses part of the “input” recognition sequence and the ribosome recognition sequence, which is kept inaccessible. The toehold switch is turned on when a stimulating “input” RNA binds to the toehold and induces the hairpin structure to open up, giving the ribosome access to its recognition sequence to start the synthesis of the encoded protein downstream, which can generate a detectable signal.

In another example, a quenched fluorophore is added to the substrate, which becomes released and thus emits fluorescence once the substrate is cut, thus enabling target detection.

The methods here can be used to detect or quantitate different types of nucleic acids, such as a single strand RNA, a double stranded RNA, a single strand DNA or a double strand DNA. The nucleic acid may be from any types of samples, such as a clinical sample suspected of infection, or a sample requiring mutation or SNP (single nucleotide polymorphism) detection, without limitation.

### Compositions and Kits for Carrying Out the Methods

Compositions and kits are also provided that can be used for carrying out the methods of the present disclosure.

In one embodiment, provided is a kit, package, or composition, for detecting a target polynucleotide. In some embodiments, the kit, package or composition includes (a) a polymerase, (b) deoxynucleoside triphosphates (dNTPs), (c) primers for amplifying the target polynucleotide, (c) a CRISPR-associated (Cas) nuclease, and (d) a guide RNA comprising a spacer fragment complementary to a target fragment on the target polynucleotide. These ingredients are present such that the polymerase can effectively amplify the target polynucleotide while the Cas nuclease is capable of cleaving the amplified target polynucleotide. In some embodiments, target fragments amplified by polymerase include suboptimal or non-canonical PAM sequences targeted by a guide RNA and a Cas nuclease.

In another embodiment, the kit, package or composition includes (a) a polymerase, (b) deoxynucleoside triphosphates (dNTPs), and (c) primers for amplifying the target polynucleotide, wherein the dNTPs and/or the primers are modified/substituted such that the amplified products have reduced binding to a CRISPR system, such as those described herein. In some embodiments, the primer(s) includes a PAM sequence for a Cas nuclease. In some embodiments, the PAM sequence is a suboptimal or non-canonical PAM sequence.

Another embodiment provides a kit, package or composition for cleaving a target polynucleotide, which includes (a) a CRISPR-associated (Cas) nuclease, and (b) a guide RNA comprising a spacer fragment complementary to a target fragment on the target polynucleotide, wherein the guide RNA, as compared to a standard guide RNA, has reduced binding to or cleaving of the target polynucleotide.

Also provided, in another embodiment, is a mutant Cas nuclease having (a) reduced activity in forming a ribonucleoprotein (RNP), (b) changed conformation, (c) reduced activity in interacting with a target PAM sequence, or (d) reduced binding to a target polynucleotide to be cleaved.

In some embodiments, the polymerase is one that is capable of, together with the dNTP and primers, amplification of the target polynucleotide. In some embodiments, the amplification is isothermal amplification. Example polymerases and corresponding isothermal amplification systems are described above.

In some embodiments, one or more of the dNTPs are modified. In some embodiments, the modification leads to reduced binding or cleavage by the Cas nuclease. Examples of such modifications are also provided herein. The suitable percentage of such modifications are also described herein.

In some embodiments, one or more nucleotides in one or more of the primers are modified. In some embodiments, the modification leads to reduced binding or cleavage by the Cas nuclease. Examples of such modifications are also provided herein. The suitable percentage of such modifications are also described herein.

In some embodiments, the primers and/or guide RNA are designed such that a suboptimal PAM sequence is included in the amplified sequence for targeting by the guide RNA/Cas nuclease. Examples of such suboptimal PAM sequences and their corresponding Cas nucleases are also described herein.

In some embodiments, the guide RNA is designed such that its binding to the Cas nuclease or the target polynucleotide is reduced. Such design includes truncation, extension, modification, without limitation. Examples are also provided herein.

In some embodiments, sequence engineered Cas nucleases are provided that have reduced binding to the guide RNA or the target polynucleotide, or reduced cleavage of the target polynucleotide. Examples residues for such mutations and example mutations are also provided in the instant disclosure.

The methods and compositions of the instant disclosure are useful for quick and efficient detection of nucleic acids, such as clinical samples with potential viral infections, genomic DNA with potential SNP (single nucleotide polymorphism), without limitation. As demonstrated, the instant application provides simple, instrument-free and sensitive alternatives to gold-standard PCR, and holds the great potential to enable rapid, point-of-care screening for nucleic acid molecules of interest.

### EXAMPLES

### Example 1: Accelerated One-Pot Test with Enhanced Sensitivity, Reliability and Flexibility Using Suboptimal PAM of Cas12a

This example demonstrates a Flexible, Accelerated, Suboptimal PAM-based Test with Enhanced sensitivity and Reproducibility (FASTER) detection method. This example found that, in the one-step CRISPR detection whereas isothermal amplification and cleavage of Cas12a occurred simultaneously in the same tube, crRNA targeting substrates with suboptimal PAM rather than routinely used canonical PAM could accelerate the reaction speed by 2-3 folds. Moreover, vast tests demonstrated that the FASTER detection had greater sensitivity and reliability due to less disturbance of isothermal amplification from Cas12a. The much higher prevalence of suboptimal PAM makes development of detection kit more flexible to optimize. FASTER detection allowed to detect a DNA virus human cytomegalovirus as little as 8 minutes, and the RNA virus SARS-CoV-2 in 15 minutes, with comparable limit of detection to qPCR in both cases. Due to its fast turnaround time, high sensitivity and reliability, FASTER detection holds great potential to facile developing point-of-care diagnostic.

### Materials and Methods

Plasmid and dsDNA Preparation

The envelope (E) and spike (S) genes of SARS-CoV-2 were synthesized and cloned into the pUC57 vector (GenScript Biotech, Nanjing, China). The N gene dsDNA of SARS-CoV-2 was obtained by RT-PCR using inactivated viruses, and N gene dsDNA of other human coronaviruses were synthesized (GenScript Biotech, Nanjing, China). The Orflab dsDNA substrates containing spacer 4 and spacer 5 targeting regions were obtained by PCR. UL55 dsDNA was obtained by PCR using inactivated HCMV virus as a template and cloned into the pUC57 vector. The SARS-CoV-2 Pseudovirus was lentivirus packaged with SARS-CoV-2 N gene (Beyotime Biotechnology, Shanghai, China).

### Preparation of HCMV

Viral samples were collected from the supernatant of cells cultured after infection with HCMV. The HCMV viral sample was inactivated at 95° C. and diluted 1:1 in lysis buffer (QuickExtract DNA Extraction Solution, Lucigen, USA). The copy number was quantified by qPCR according to the standard curve generated using plasmid DNA.

### LbCas12a Protein Expression and Purification

The DNA fragment encoding LbCas12a was cloned into a pET-based expression vector containing a C-terminal 6×His-tag. E. coli strain BL21 (DE3) transformed by the recombinant plasmid was incubated with 0.5 mM isopropyl β-D-1-thiogalactopyranoside (IPTG) when the culture density reached an OD₆₀₀ of 0.7, and was grown at 21° C. for another 16 hours. The proteins were purified from the cell lysate via Ni-NTA resin and eluted with buffer (20 mM Tris-HCl, 500 mM NaCl and 500 mM imidazole, pH 7.4). Then, the concentrated protein was further filtered using a gel filtration column (Superdex 200 Increase 10/300 GL) in elution buffer containing 20 mM Tris-HCl, pH 7.5, 200 mM NaCl, and the final storage buffer comprised by 20 mM Tris-HCl, pH 7.5, 200 mM NaCl, 5% glycerol.

### RPA and RT-RPA

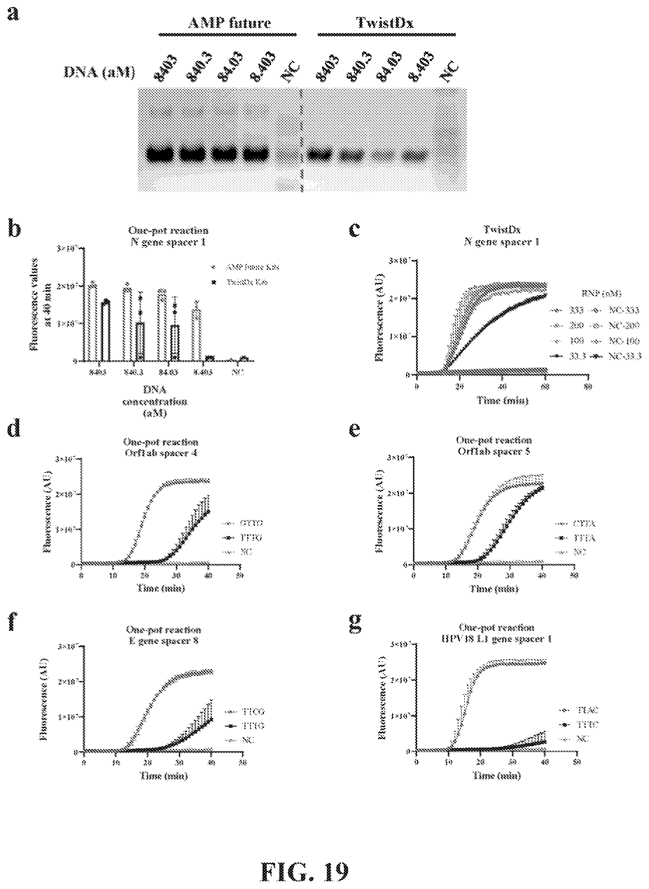

One lyophilized RPA pellet was resuspended in 29.4 μL Buffer A, 16.1 μL nuclease-free water, 1 μL of 20 μM RPA forward primer, and 1 μL of 20 μM RPA reverse primer to form the RPA mix according to the manufacturer's instructions (Weifang Amp-Future Biotech, Shandong, China). RPA kits from TwistDx (Product code: TABAS03KIT) were used in FIG. 19. TwistDx RPA mix was resuspended in 29.5 μL rehydration buffer, 15.6 μL nuclease-free water, 1.2 μL of 20 μM RPA forward primer, and 1.2 μL of 20 μM RPA reverse primer. The primer sequences are presented in Table 1. For the RT-RPA reaction, 0.9 μL RNase H (50 U/μL stock, New England Biolabs, USA) and 0.45 μL SuperScript IV reverse transcriptase (Thermo Fisher Scientific, USA) or EpiScript RNase H-Reverse Transcriptase (Lucigen) were added to the RPA mixture (PMID: 32848209; 33219228) The reactions were performed at 37° C. or 42° C. In finalized version of RT-RPA reactions, 1 μL of 5 μM RPA reverse primer and an additional RT primer (1 μL of 40 μM) was added, and mixed thoroughly.

TABLE 1
RPA and RT-RPA primers

| Name | Sequence | SEQ ID NO: |
|---|---|---|
| Orflab spacer 4 RPA-F | CTAAAGCTTACAAAGATTATCTAGCTAGTGG | 1 |
| Orflab spacer 4 RPA-R | TTTGTACATACTTACCTTTTAAGTCACAAAATC | 2 |
| Orflab spacer 5 RPA-F | CTAAAGCTTACAAAGATTATCTAGCTAGTGG | 3 |
| Orflab spacer 5 RPA-R | TTTGTACATACTTACCTTTTAAGTCACAAAATC | 4 |
| S gene spacer 1 RPA-F | AGGTTTCAAACTTTACTTGCTTTACATAGA | 5 |
| S gene spacer 1 RPA-R | TCCTAGGTTGAAGATAACCCACATAATAAG | 6 |
| S gene spacer 2 RPA-F | AGGTTTCAAACTTTACTTGCTTTACATAGA | 7 |
| S gene spacer 2 RPA-R | TCCTAGGTTGAAGATAACCCACATAATAAG | 8 |
| S gene spacer 3 RPA-F | AGGTTTCAAACTTTACTTGCTTTACATAGA | 9 |
| S gene spacer 3 RPA-R | GCATCTGTAATGGTTCCATTTTCATTATATT | 10 |
| E gene RPA-F | ATGTACTCATTCGTTTCGGAAGAGACAGGTA | 11 |
| E gene RPA-R | TTAGACCAGAAGATCAGGAACTCTAGAAGAATT | 12 |
| HPV18 L1 RPA-F | ATGATTATGTGACTCGCACAAGCATATTTTAT | 13 |
| HPV18 L1 RPA-R | ACTAAACGTTGTGTTTCAGGATTATAAATACT | 14 |
| HCMV5 UL55 RPA-F | TAGCTACGCTTACATCTACACCACTTATC | 15 |
| HCMV5 UL55 RPA-R | TAGGAACTGTAGCATTGAGCAAACTTGTTGATG | 16 |
| N gene spacer 1 RPA-F | TTCCCTCGAGGACAAGGCGTTCCAATTAA | 17 |
| N gene spacer 1 RPA-R | TTCAAGGCTCCCTCAGTTGCAACCCATATGAT | 18 |

Preparation of crRNA

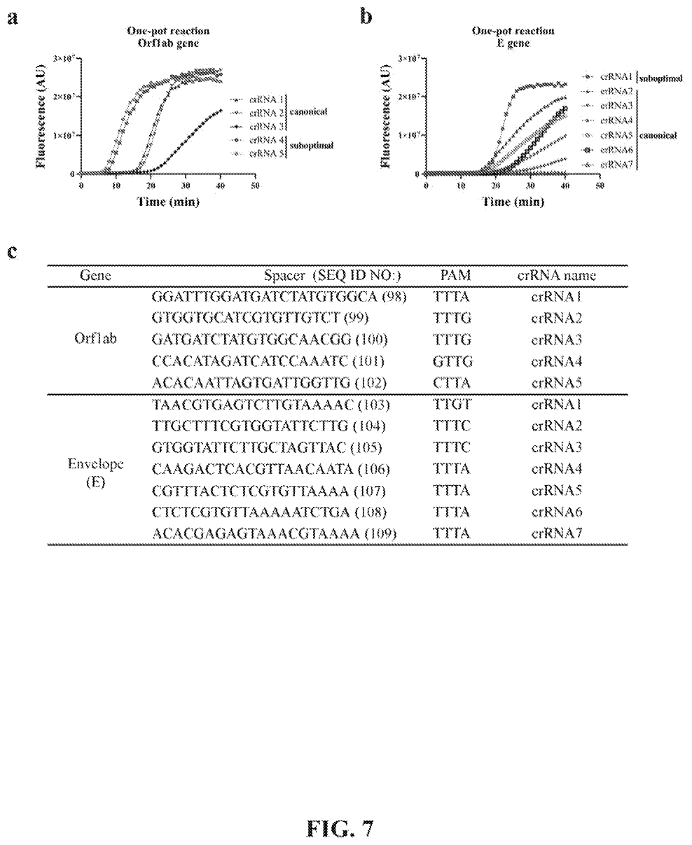

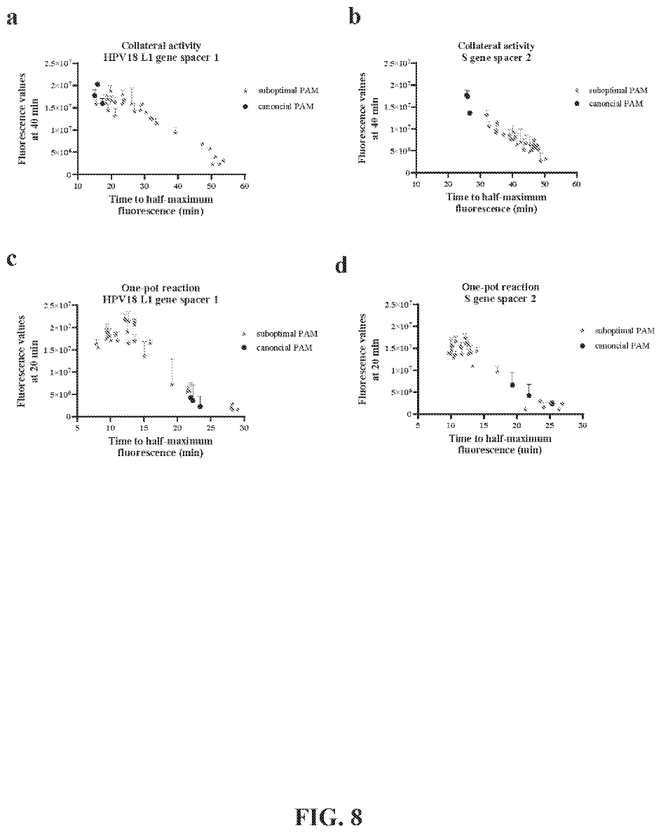

The DNA template for in vitro transcription was synthesized by overlapping PCR of two oligos. One oligo contained the T7 promotor sequence and the other contained spacer sequence. The PCR product was incubated with T7 RNA polymerase for in vitro transcription at 37° C. for 2 h. The IVT reaction was treated with DNase I (Promega) for 15 min at 37° C., and then purified using Monarch RNA Cleanup Kit (NEB). The sequences of crRNA were presented in FIG. 7c and Table 2.

TABLE 2
crRNA sequence

| Name | crRNA | SEQ ID NO: |
|---|---|---|
| Orflab spacer 4 | UAAUUUCUACUAAGUGUAGAUCCACAUAGAUCA | 19 |
| | UCCAAAUC | |
| Orflab spacer 5 | UAAUUUCUACUAAGUGUAGAUACACAAUUAGUG | 20 |
| | AUUGGUUG | |
| Spike spacer 1 | UAAUUUCUACUAAGUGUAGAUGACAGCUGGUGC | 21 |
| | UGCAGCUU | |
| Spike spacer 2 | UAAUUUCUACUAAGUGUAGAUUCAGGUUGGACA | 22 |
| | GCUGGUGC | |
| Spike spacer 3 | UAAUUUCUACUAAGUGUAGAUUGUGGGUUAUCU | 23 |
| | UCAACCUA | |
| E gene | UAAUUUCUACUAAGUGUAGAUAUUGUGUGCGUA | 24 |
| | CUGCUGCA | |
| HPV18 L1 | UAAUUUCUACUAAGUGUAGAUCAACAGUUAAUA | 25 |
| | AUCUAGAG | |
| HCMV5 UL55 | UAAUUUCUACUAAGUGUAGAUUGUGAUGAAUCU | 26 |
| | CCCACAUA | |
| N gene spacer 1 | UAAUUUCUACUAAGUGUAGAUUCAUCUGGACUG | 27 |
| | CUAUUGGU | |

### One-Pot Assay

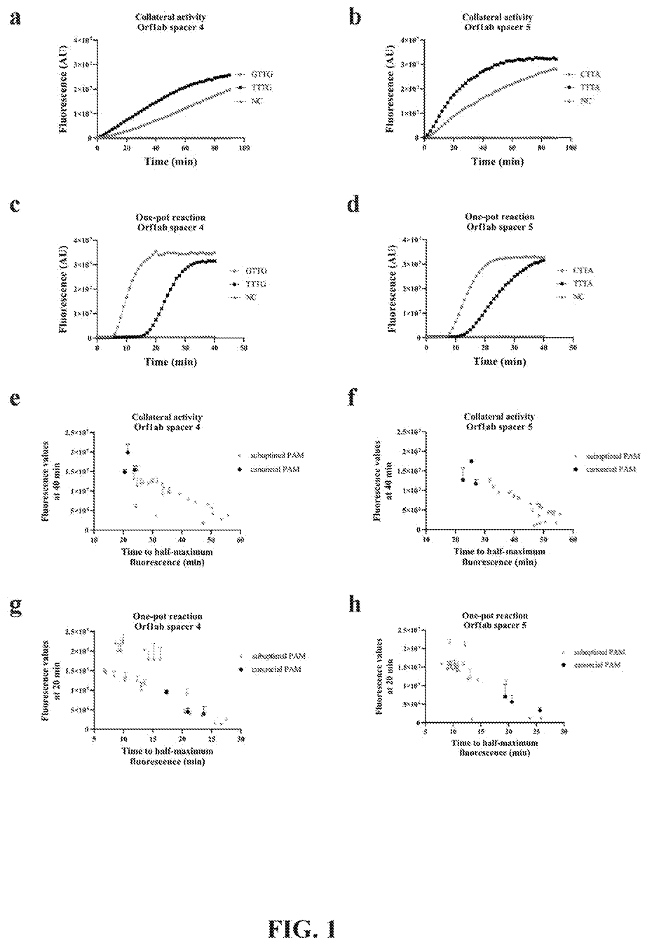

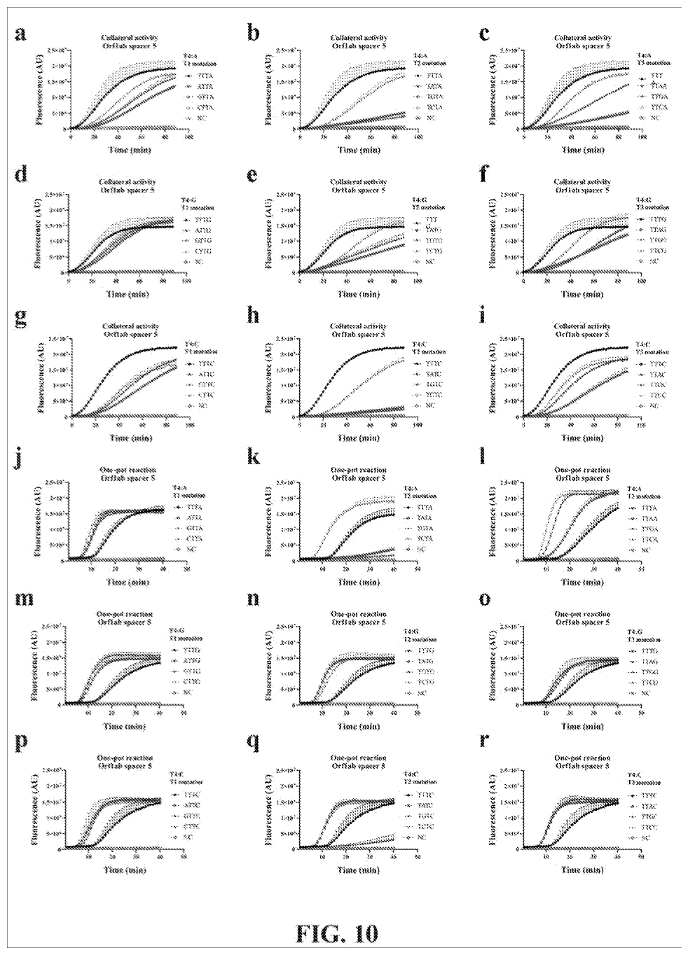

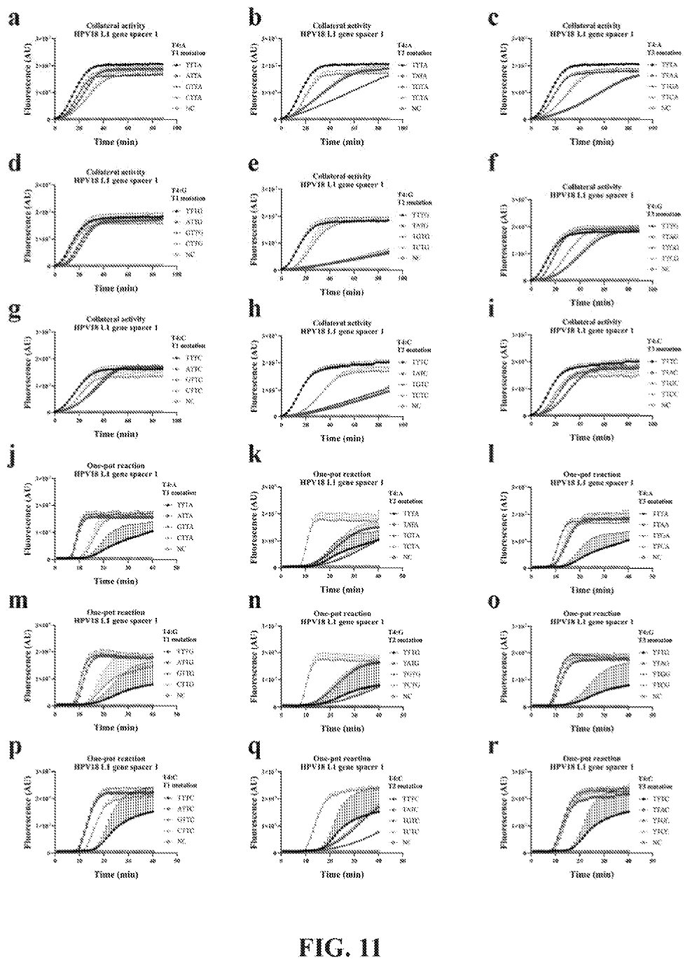

One-pot assays were performed in 30 μL reaction volume containing 33 or 100 nM LbCas12a RNP, 400 nM FQ ssDNA reporter (FAM-TTATT-Quencher, Takara Biotechnology), dsDNA substrate (Table 3) and RPA or RT-RPA components in plate wells (Corning, USA). The RNP complex, FQ ssDNA reporter (8 μL) and RPA mixture (18 μL) were added to each one-pot reaction well, and subsequently, 2 μL of Buffer B and dsDNA activator were supplied prior to read out through a SpectraMax i3x at 37° C. or 42° C. The assay was also monitored under UV, blue light or by lateral flow detection (Milenia HybriDetect 1 kit, TwistDx, United Kingdom). The final concentration of reporter for UV detection was adjusted to 0.4-2 μM. The reporter for lateral flow detection was FAM-TTATTATT-Biotin with a final concentration of 800 nM. The concentration of dsDNA substrate used was 18.3 fM-2.3 pM for FIG. 1, FIGS. 8-12 & 14.

TABLE 3
PAM and target sequence

| | Sequence (non-target and target | |
|---|---|---|
| Name | strands containing PAM) | SEQ ID NO: |
| Orflab spacer 4 target | NTS: 5′-GTTGCCACATAGATCATCCAAATC-3′ | 28 |
| | TS: 3′-CAACGGTGTATCTAGTAGGTTTAG-5′ | 29 |
| Orflab spacer 5 target | NTS: 5′-CTTAACACAATTAGTGATTGGTTG-3′ | 30 |
| | TS: 3′-GAATTGTGTTAATCACTAACCAAC-5′ | 31 |
| Spike spacer 1 target | NTS: 5′-GTTGGACAGCTGGTGCTGCAGCTT-3′ | 32 |
| | TS: 3′-CAACCTGTCGACCACGACGTCGAA-5′ | 33 |
| Spike spacer 2 target | NTS: 5′-TTCTTCAGGTTGGACAGCTGGTGC-3′ | 34 |
| | TS: 3′-AAGAAGTCCAACCTGTCGACCACG-5′ | 35 |
| Spike spacer 3 target | NTS: 5′-ATTATGTGGGTTATCTTCAACCTA-3′ | 36 |
| | TS: 3′-TAATACACCCAATAGAAGTTGGAT-5′ | 37 |
| E spacer 8 target | NTS: 5′-TTCGATTGTGTGCGTACTGCTGCA-3′ | 38 |
| | TS: 3′-AAGCTAACACACGCATGACGACGT-5′ | 39 |
| HPV18 L1 target | NTS: 5′-TTACCAACAGTTAATAATCTAGAG-3′ | 40 |
| | TS: 3′-AATGGTTGTCAATTATTAGATCTC-5′ | 41 |
| HCMV5 UL55 target | NTS: 5′-TTGATGTGATGAATCTCCCACATA-3′ | 42 |
| | TS: 3′-AACTACACTACTTAGAGGGTGTAT-5′ | 43 |
| N gene spacer 1 target | NTS: 5′-TTGGTCATCTGGACTGCTATTGGT-3′ | 44 |
| | TS: 3′-AACCAGTAGACCTGACGATAACCA-5′ | 45 |

### Deep Sequencing

The deep sequencing samples were prepared as one pot detection reactions, except that substrate was mixed by canonical-PAM and suboptimal-PAM substrates at 1:1 ratio. The reaction was terminated by adding proteinase K (Thermo Fisher scientific) at different time points, and then heated at 95° C. for 5 minutes to inactivate the protease. The products were amplified with adapters and barcode (Table 4) for NovaSeq of Illumina, and the resulting reads were filtered by an average Phred quality (Q score) at least 25. Raw reads were analyzed by Python Scripts and data was normalized according to reads of 0-minute time point.

TABLE 4
Deep sequencing primers

| Name | Sequence (capital letters represent barcode) | SEQ ID NO: |
|---|---|---|
| F1 | ggagtgagtacggtgtgcAATTTctaaagcttacaaagattat | 46 |
| F2 | ggagtgagtacggtgtgcCTATTctaaagcttacaaagattat | 47 |
| F3 | ggagtgagtacggtgtgcCGATTctaaagcttacaaagattat | 48 |
| F4 | ggagtgagtacggtgtgcAGGCGctaaagcttacaaagattat | 49 |
| F5 | ggagtgagtacggtgtgcTCCTCctaaagcttacaaagattat | 50 |
| F6 | ggagtgagtacggtgtgcACTAActaaagcttacaaagattat | 51 |
| F7 | ggagtgagtacggtgtgcGTGGCctaaagcttacaaagattat | 52 |
| F8 | ggagtgagtacggtgtgcATAAActaaagcttacaaagattat | 53 |
| F9 | ggagtgagtacggtgtgcCACGCctaaagcttacaaagattat | 54 |
| F10 | ggagtgagtacggtgtgcGTAGCctaaagcttacaaagattat | 55 |
| F11 | ggagtgagtacggtgtgcGAAGTctaaagcttacaaagattat | 56 |
| F12 | ggagtgagtacggtgtgcCTGTGctaaagcttacaaagattat | 57 |
| F13 | ggagtgagtacggtgtgcACCCActaaagcttacaaagattat | 58 |
| F14 | ggagtgagtacggtgtgcGGGTGctaaagcttacaaagattat | 59 |
| F15 | ggagtgagtacggtgtgcGAGATctaaagcttacaaagattat | 60 |
| F16 | ggagtgagtacggtgtgcGCGCGctaaagcttacaaagattat | 61 |
| R1 | gagttggatgctggatggACAAGtttgtacatacttacctttt | 62 |
| R2 | gagttggatgctggatggTGGCTtttgtacatacttacctttt | 63 |
| R3 | gagttggatgctggatggTGCCCtttgtacatacttacctttt | 64 |
| R4 | gagttggatgctggatggATTTCtttgtacatacttacctttt | 65 |
| R5 | gagttggatgctggatggCCCTGtttgtacatacttacctttt | 66 |
| R6 | gagttggatgctggatggTCATTtttgtacatacttacctttt | 67 |
| R7 | gagttggatgctggatggGCGTAtttgtacatacttacctttt | 68 |
| R8 | gagttggatgctggatggCCGCAtttgtacatacttacctttt | 69 |
| R9 | gagttggatgctggatggACACGtttgtacatacttacctttt | 70 |
| R10 | gagttggatgctggatggCTTCGtttgtacatacttacctttt | 71 |
| R11 | gagttggatgctggatggGAGCGtttgtacatacttacctttt | 72 |
| R12 | gagttggatgctggatggGTACGtttgtacatacttacctttt | 73 |
| R13 | gagttggatgctggatggGTTTAtttgtacatacttacctttt | 74 |
| R14 | gagttggatgctggatggCGCTCtttgtacatacttacctttt | 75 |
| R15 | gagttggatgctggatggTTGAAtttgtacatacttacctttt | 76 |
| R16 | gagttggatgctggatggGATCGtttgtacatacttacctttt | 77 |

Fn and Rn represent a pair of primers.

### Cas12a In Vitro Cleavage and Collateral Activity

For in vitro cleavage, the LbCas12a RNP was incubated at room temperature for 20 minutes in 1×NEBuffer 2.1 prior to incubation with dsDNA at 37° C. The reaction was terminated by adding proteinase K at various time points, and the product were visualized on a 2% TAE gel. The concentrations of RNP used were 50 or 100 nM, and the concentrations of dsDNA substrate were 6-7.5 nM or 9-11 nM. The percentage of substrates and products were quantified by Image Lab software (Bio-Rad). The cleavage efficiency at each time point was plotted as a function of time, and these data were fit with a one phase exponential decay curve, to calculate K_{cleave} values (Prism 8, GraphPad Software, Inc.) (PMID: 26545076). The collateral activity assay was performed in a 30 μL volume containing 33 nM LbCas12a RNP, and 400 nM ssDNA reporter (FAM-TTATT-BHQ1) in 1×NEBuffer 2.1, and the fluorescence signal was recorded by SpectraMax i3x. The concentrations of dsDNA substrate activators used were 2.7-3.5 nM.

### EMSA

Deactivated LbCas12a (D832A) (briefly as dCas12a) was expressed and purified as described above. An electrophoretic mobility shift assay was performed with dLbCas12a RNP and a 5′-FAM labeled 50-nt dsDNA substrate using 1×NEBuffer 2.1. Binding was carries out at 37° C. for 15 minutes and then the reactions were supplemented with 5% glycerol. Samples were then resolved on 4% Tris-borate/EDTA polyacrylamide gels for 15-20 minutes at a voltage of 120V, and the results were visualized by a fluorescent image analyzer.

qPCR and RT-qPCR Assay

qPCR assays for HCMV samples were performed in a 20 μL reaction volume containing 10 μL of 2×AceQ qPCR Probe Master Mix (Vazyme, Nanjing, China), 1 μL of each primer pair at 10 μM (Table 5) and 0.2 μL of 10 μM TaqMan probe (GenScript, China). The numbers of viral copies input and sample processing in qPCR and FASTER were the same. Each RT-qPCR reaction for SARS-CoV-2 samples contained 10 μL of 2×One Step SYBR Green Mix, 1 μL of One Step SYBR Green Enzyme Mix (Vazyme, China), 0.4 μL of the primer pairs at 10 μM. The input volume of RT-qPCR assay was 1.34 μL sample per 20 μL reaction.

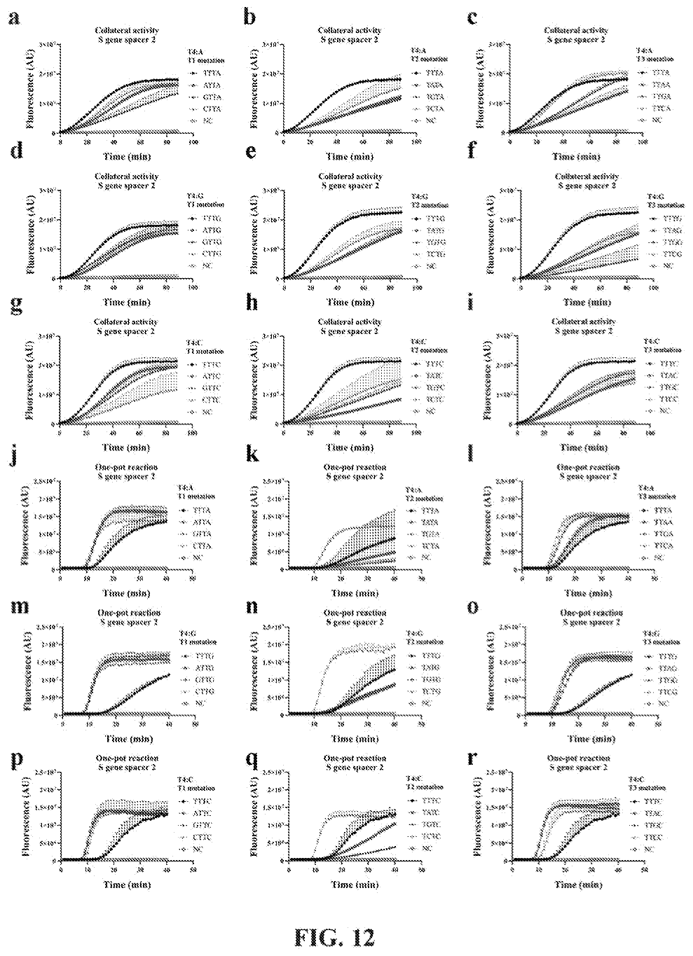

TABLE 5
qPCR primers, probe sequence and PCR primers and RT primers

| Name | Sequence | SEQ ID NO: |
|---|---|---|
| qPCR UL55 F | CTACCCTCAAGTACGGAGATGTG | 78 |
| qPCR UL55 R | GTCTTCATTGATAGGCTTCATCG | 79 |
| qPCR UL55 Probe | FAM-ATCTTATTCGCTTTGAACGTAATATCAT-BHQ1 | 80 |
| CDC RT-qPCR N2-F | TTACAAACATTGGCCGCAAA | 81 |
| CDC RT-qPCR N2-R | GCGCGACATTCCGAAGAA | 82 |
| Orflab spacer 4 PCR primers | F: TGGAACCACCTTGTAGGTTTG | 83 |
| for in vitro cleavage | R: TATGCACCACCGGGTAAAGT | 84 |
| Orflab spacer 5 PCR primers | F: TGGAACCACCTTGTAGGTTTG | 85 |
| for in vitro cleavage | R: ACCCACAGGGTCATTAGCAC | 86 |
| Orflab spacer 4, 5 PCR | F: TGGAACCACCTTGTAGGTTTG | 87 |
| primers for collateral activity | R: ACCCACAGGGTCATTAGCAC | 88 |
| and one-pot reaction | | |
| HPV 18 L1 gene spacer 1 | F: ACACATTATTATTTGTGGCCATT | 89 |
| PCR primer for cis-cleavage | R: GTGCCTTTAGCCCAGTGTTC | 90 |
| Orflab spacer 4 PCR primer | F: TGGAACCACCTTGTAGGTTTG | 91 |
| for cis-cleavage in | R: TTCGCGGAGTTGATCACAACTA | 92 |
| Supplementary FIG. 12 | | |
| S gene spacer 2 PCR primer | F: TTGGATGGAAAGTGAGTTCAGA | 93 |
| for cis-cleavage | R: ACTTCACCAAAAGGGCACAA | 94 |
| N gene RT-1 | ATGTGATCTTTTGGTGTA | 95 |
| N gene RT-2 | ATCTTTTGGTGTATTCAA | 96 |
| N gene RT-3 | TTGCAGCATTGTTAGCA | 97 |

### SARS-CoV-2 Clinical Sample Collection

The clinical samples used in this study were approved by the Wuhan Jinyintan Hospital Ethics Committee (KY-2021-01.01). SARS-CoV-2-positive and -negative samples were obtained from Wuhan Jinyintan Hospital. Unextracted samples were lysed at 95° C. for 5-10 minutes with an equal volume of lysis buffer containing 1 U/μL RNasin Plus, 250 μM TCEP and 0.02 μg/μL Chelex-100 (PMID: 32577657; 32958655). The extracted RNA samples were purified according to the manufacture's protocol (liferiver, Shanghai). They were mixed with RT primer for FASTER detection. The UV images for all samples were processed in Image Lab (Bio-Rad) under these parameters: time of exposure: 0.368-0.636, Gamma value: 0.9-1.14. STOPCovid.v1 assay was performed exactly following the protocol (PMID: 32937062).

### Results

Here we described a fast and simple CRISPR-based diagnostic to detect DNA and RNA viruses, including SARS-CoV-2. First, we designed a number of crRNAs targeting the ORF lab and E genes of SARS-CoV-2, and performed one-pot test in which the recombinase polymerase amplification (RPA) and Cas12a-based detection were combined together in one reaction. We noticed that several crRNAs displayed faster kinetics of fluorescence signals than others in the one-pot reaction (FIG. 7a-b). Detailed analysis of outperformed crRNAs, we found those crRNAs were all designed to use suboptimal PAM of Cas12a (NTTV and TTNT) rather than the canonical PAM (TTTV) (FIG. 7c). Based on these observations, we hypothesized that crRNAs using suboptimal PAM accelerated the speed of detection in one-pot test, where isothermal amplification of target and Cas12a-mediated cleavage of target occurred simultaneously. To test this, the substrates of spacer 4 and 5 targeting ORF lab gene were point-mutated to convert their suboptimal PAM to the canonical PAM. As expected, crRNA using suboptimal PAM generated weaker and slower collateral activity than the canonical PAM (FIG. 1a-b). We then used these substrates as the RPA template, and performed RPA and Cas12a-based detection simultaneously in one-pot test. In contrast to the collateral activity assay, both spacer 4 and 5 using suboptimal PAM exhibited faster kinetics than the canonical PAM in one-pot reaction (FIG. 1c-d).

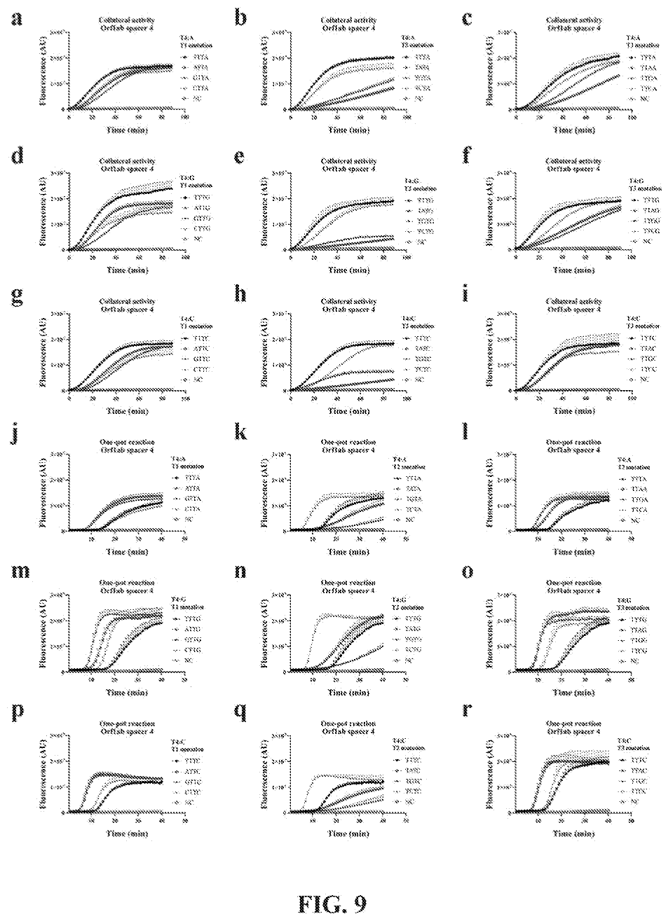

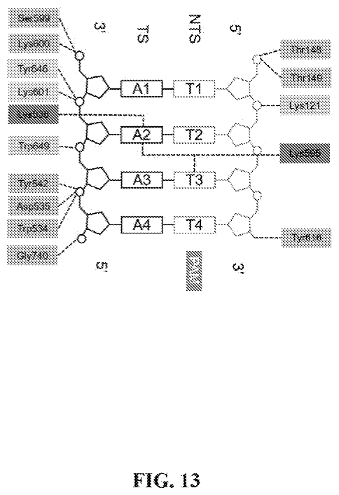

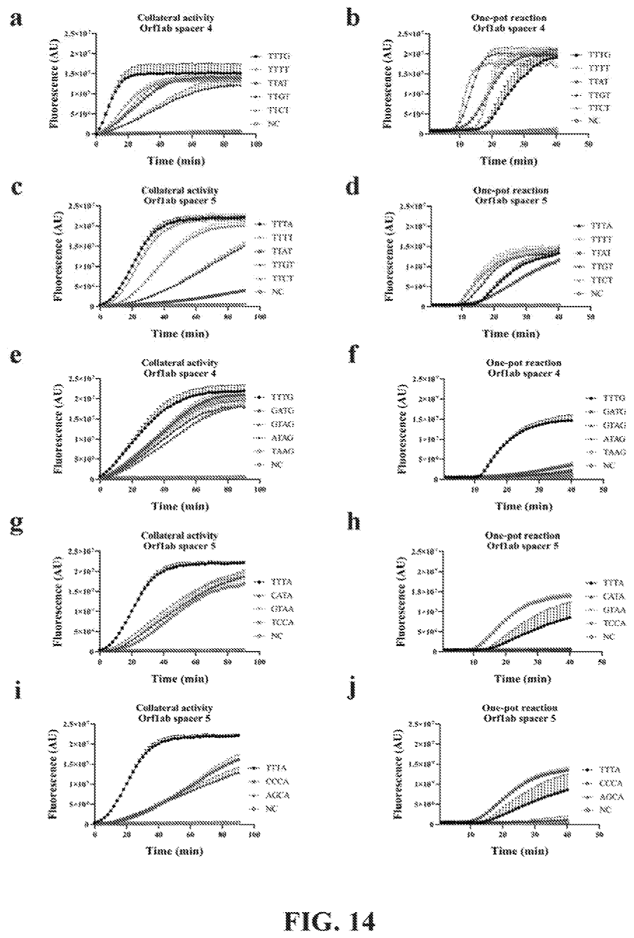

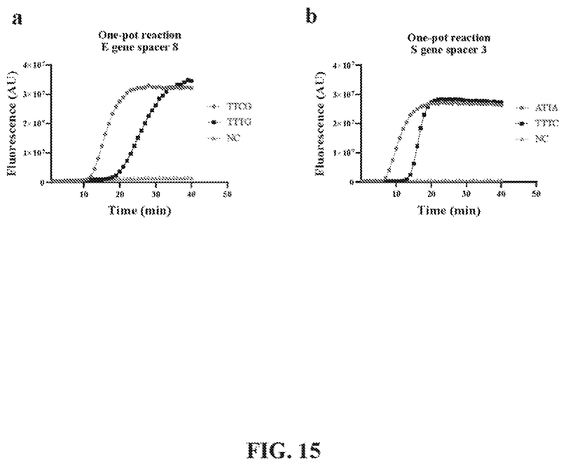

To explore which types of suboptimal PAMs exhibited faster reactions in the one-pot reactions, substrates of spacers 4 and 5 of the Orflab gene, spacer 2 of Spike (S) gene of SARS-CoV-2 and spacer 1 of the HPV18 L1 gene were point-mutated from TTTV to VTTV, TVTV, or TTVV. A comparison of the collateral activity and one-pot reaction for 120 suboptimal PAMs of four spacers indicated that more than 80% of spacers with suboptimal PAMs showed a faster reaction than those with the canonical PAM in the one-pot reaction, and most of the outperforming suboptimal PAMs were VTTV, TCTV and TTVV (FIG. 1e-h, FIG. 8-12). The protein structure of Cas12a shows that the PAM-interacting domain mainly contacts the second nucleotide of the target strand; therefore, mutating the second nucleotide of PAM from pyrimidine to purine is likely to dramatically impair the activity of Cas12a (FIG. 13). Indeed, some TATV and TGTV PAMs, but not TCTV PAMs, showed slower kinetics with reduced fluorescence signals in the one-pot reaction; and consistently, these suboptimal PAMs all demonstrated much lower collateral activity than the canonical PAMs (FIG. 9-12). For spacer 4 and 5, TTTT PAM exhibited faster kinetics than TTTV PAM in the one-pot reaction, indicating that the fourth nucleotide of the PAM may also be modified to tune the activity of Cas12a (FIG. 14a-d). We then introduced two point mutations into the PAM. The introduction of two PAM point mutations (TTTV to TTVT) for spacer 4 and part of spacer 5, produced faster kinetics in the one-pot reaction (FIG. 14a-d). We then examined other suboptimal PAMs bearing two point mutations. The VVTV and VTVV PAMs for spacers 4 and 5 showed reduced kinetics and signals in the one-pot reaction and collateral activity (FIG. 14e-h). Interestingly, mutation of TTTV to TCCV produced a superior reaction in the one-pot reaction (FIG. 14h). This may be because the PAM of Cas12a can tolerate the T to C mutation; TCCV has been characterized as a functional suboptimal PAM(PMID: 28781234). CCCV, with three point mutations, was also recognized as a suboptimal PAM, and indeed, CCCA was faster than the canonical PAM in the one-pot reaction (FIG. 14i-j). As a negative control, another sequence with three point mutations of the PAM sequences, AGCA, showed minimal activity in the one-pot reaction. Together, it suggests that a delicate level of collateral activity is crucial in the one-pot reaction. Here we concluded that most VTTV, TCTV and TTVV, as well as some TRTV, TTNT and YYYN (except TTTV) PAMs, outperformed the canonical PAM in the one-pot reaction.

To further prove the use of suboptimal PAM sequences could accelerate the one-pot reaction, we synthesized crRNAs targeting the E and S genes of SARS-CoV-2. All these crRNAs demonstrated faster reactions on substrates with suboptimal PAM s than on those with canonical PAMs in the one-pot reaction, indicating that using suboptimal PAM could be a general strategy to accelerate the speed of the Cas12a-based one-pot test (FIG. 15).

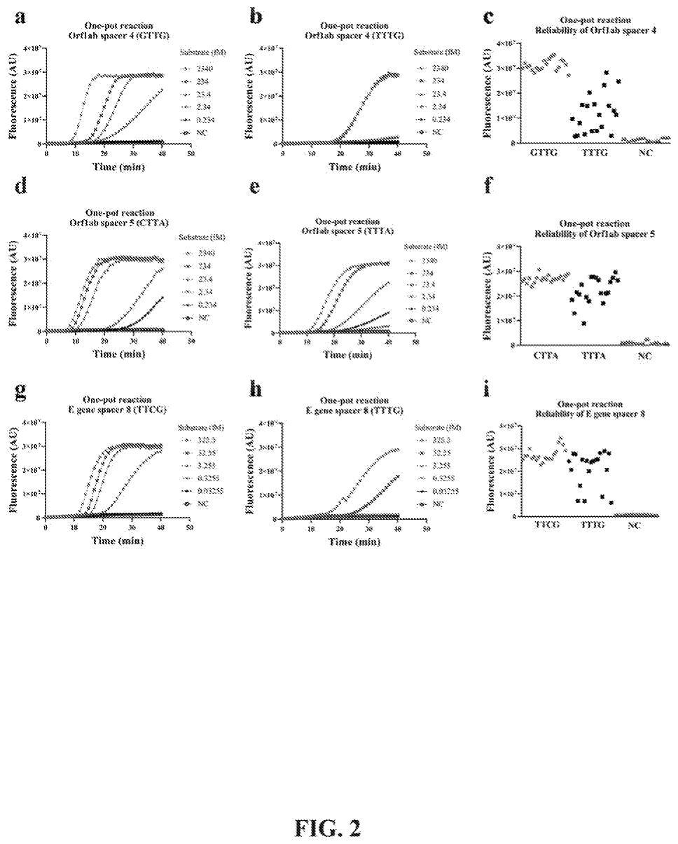

Previous studies indicate that although one-pot CRISPR diagnostics are simple to operate, their sensitivity is lower than that of two-step methods, in which target amplification and CRISPR detection are performed sequentially. We therefore investigated whether the application of suboptimal PAM could improve the sensitivity of the one-pot test. The detection limit of spacer 4 using canonical PAM was 234 fM concentration of dsDNA in the one-pot reaction; in contrast, its detection limit using suboptimal PAM was 2.34 fM concentration of dsDNA (FIGS. 2a and b). To compare the reliability of tests using suboptimal and canonical PAMs, we repeated the experiments under identical conditions ten times with two biological replicates each time. With substrates (2340 fM concentration of dsDNA) and incubation times sufficient for both suboptimal and canonical PAMs, the fluorescence signal from the suboptimal PAM group was highly consistent across all replicates; in contrast, signals from the canonical PAM group varied more than 10-fold across replicates (FIG. 2c). We then compared the detection limit and reliability of an additional two crRNAs with canonical or suboptimal PAMs. Both crRNAs using suboptimal PAMs exhibited a ˜10 to 100-fold increase in sensitivity and very consistent signal production compared with those with canonical PAMs, demonstrating the improved sensitivity and reliability of suboptimal PAM in the one-pot reaction (FIG. 2d-i).

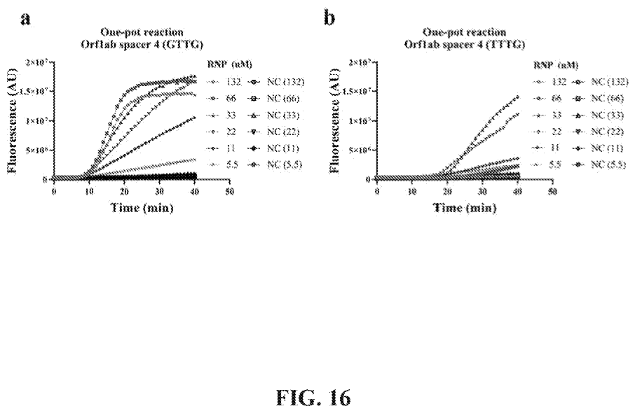

To investigate the dose effect of Cas12a/crRNA ribonucleoprotein (RNP) in the one-pot reaction, we tested RNP doses ranging from 5.5 nM to 132 nM with assays using suboptimal or canonical PAMs, respectively. Reactions with suboptimal PAM showed stable kinetic curves and consistent results with RNP dose ranging from 22-132 nM (FIG. 16a), whereas reactions with canonical PAM displayed drastic fluctuations in kinetic curves and highly variable signals, with even a minor change in RNP dose (FIG. 16b). These data further elucidate that using a suboptimal PAM is the key to reproducible results in the Cas12a-mediated one-pot test.

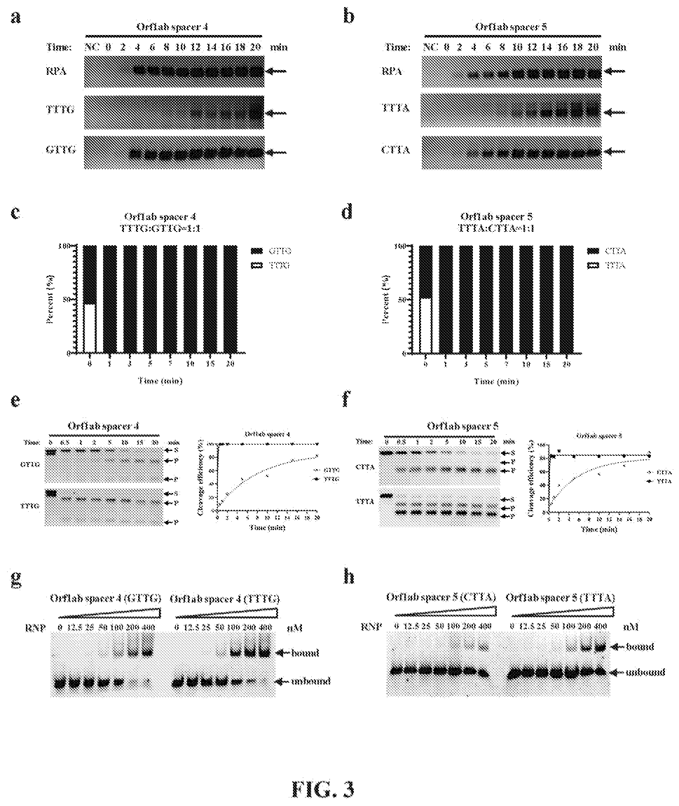

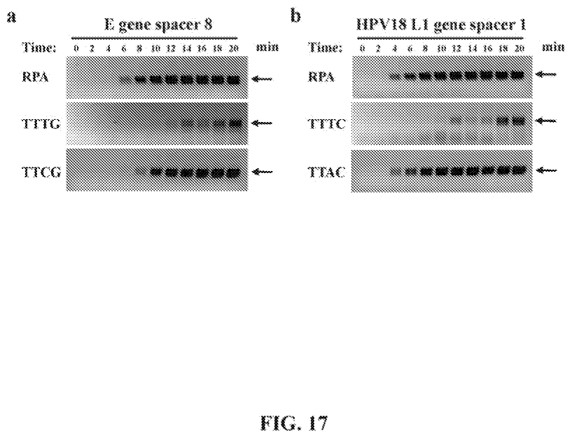

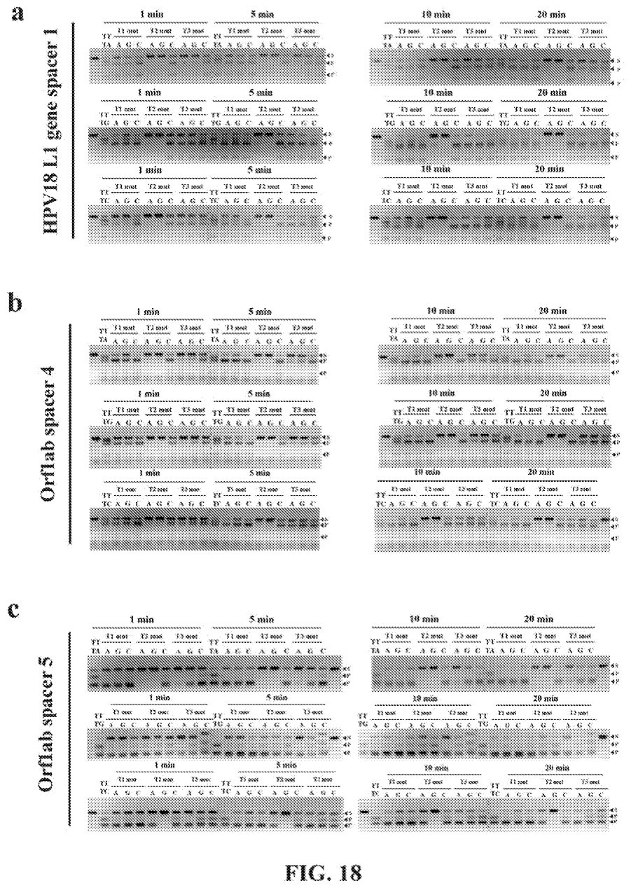

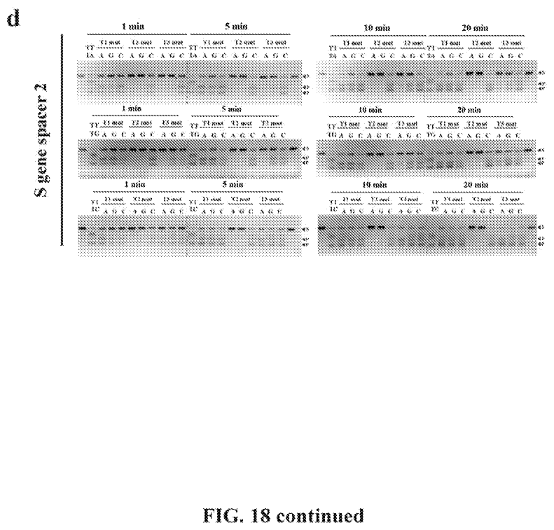

We next sought to understand the mechanism underlying the robust performance of suboptimal PAM-mediated one-pot detection. In the one-pot reaction, CRISPR detection and isothermal amplification compete against each other, and the ultimate detection signal relies on target amplification to generate adequate substrates for CRISPR detection. It is possible that crRNA using suboptimal PAM has slower initial kinetics of CRISPR detection and thus biases the reaction towards isothermal amplification. To examine this possibility, we monitored amplicon generation in the one-pot reaction. For spacer 4, the target amplicon was first observed two minutes after a one-pot reaction using suboptimal PAM, whereas in the canonical PAM group, it required eight to ten minutes to identify the amplicon (FIG. 3a). In addition, the amounts of amplicons generated by one-pot reaction with suboptimal PAM and PRA alone were much greater than that generated by one-pot reaction with canonical PAM at each time point (FIG. 3a). Consistently, the generation of amplicons by spacer 5 and two additional spacers also displayed faster kinetics and higher amounts of amplicons in tests with suboptimal PAM than those with canonical PAM, indicating a stronger interference with the RPA amplification when using canonical PAM (FIG. 3b, FIG. 17a-b). To further compare the ability of isothermal amplification under the surveillance of Cas12a, one-pot reactions were carried out using mixed substrates composed of 50% suboptimal and 50% canonical PAMs. Amplicon sequencing analysis revealed that within the first minute of reaction, the amplicon from the suboptimal PAM substrate accounts for more than ˜90% or more of the amplification products, supporting the perspective that the use of a suboptimal PAM is crucial to promote RPA amplification under the pressure of competing Cas12a cleavage (FIG. 3c-d).

Cas12a-mediated substrate binding and subsequent cis-cleavage may interfere with RPA amplification. A time course of cis-cleavage activity for a constant amount of DNA substrates showed that cleavage of the canonical PAM substrates was completed within 30 seconds, whereas it took 10-20 minutes to complete cleavage of the suboptimal PAM substrates (FIG. 3e-f). Cas12a was able to bind suboptimal PAM substrate with reduced affinity 35. We reasoned that delayed cleavage was due to weak binding of Cas12a to the DNA substrate with suboptimal PAM. In agreement, the electrophoretic mobility shift assay (EMSA) analysis of Cas12 binding affinity showed reduced binding with suboptimal PAM substrate compared for canonical PAM for both spacer 4 and 5 (FIG. 3g-h).

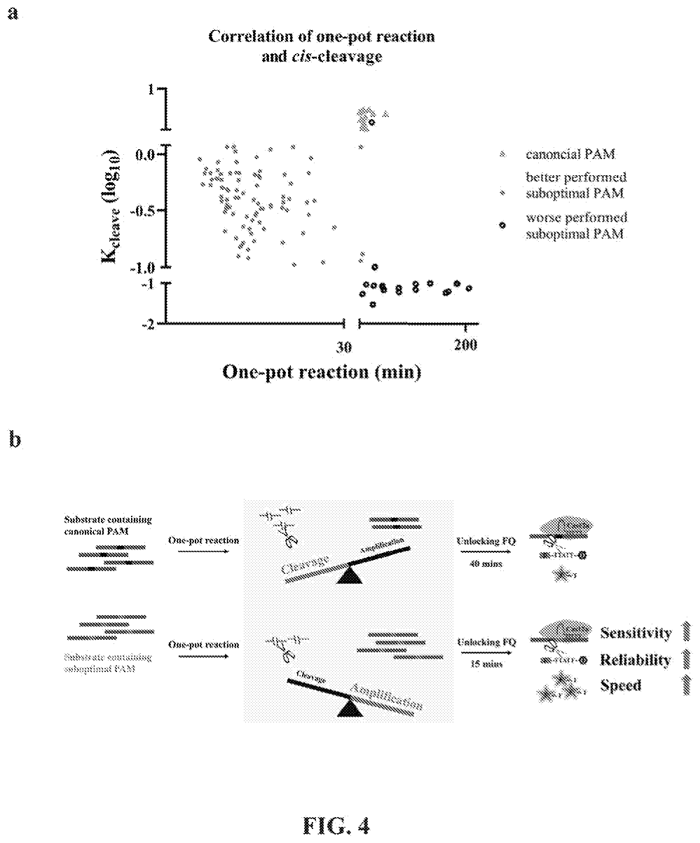

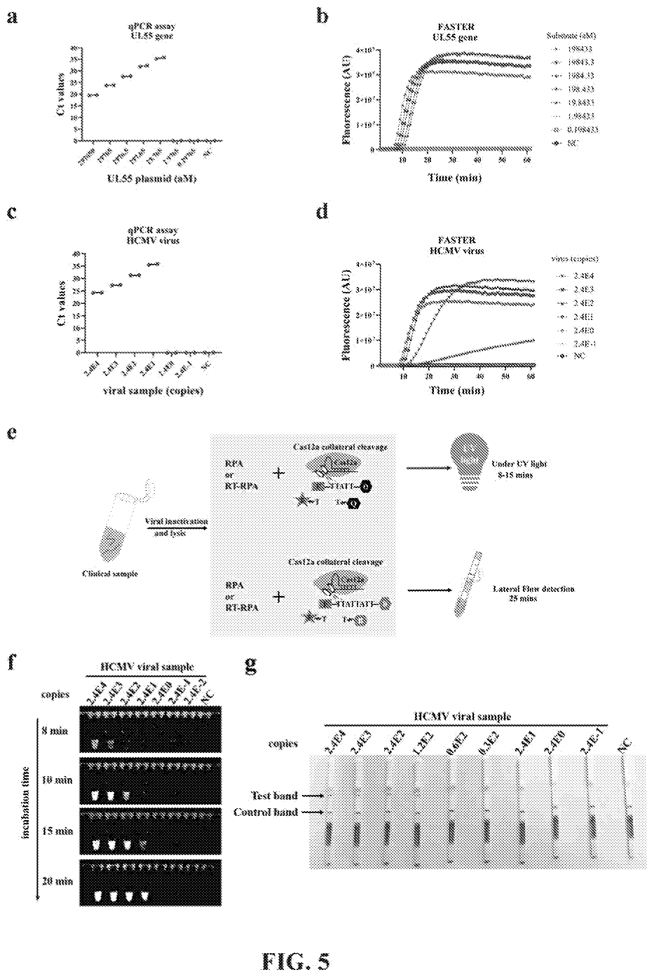

To further clarify the mechanism, we evaluated cis-cleavage activities, collateral activities and one-pot reaction of 120 PAMs (including suboptimal PAM and canonical PAM) for four different spacers (HPV18 L1 gene spacer 1, Orflab spacer 4, Orflab spacer 5 and S gene spacer 2) (FIGS. 9-12 & 18). We identified that K_{cleave} and the performance of one-pot reaction had clear correlation (FIG. 4a-b, Table 6). We define one-pot reaction factor of 30 min on the X axis as the criterion for judging whether PAM performs well in one-pot reaction. All twelve canonical PAMs of these four spacers and one suboptimal PAM of Orflab spacer 4 have high K_{cleave} of 1.2-3.5 min⁻¹, they performed well in collateral activity, but all have poor performance in one-pot reaction (FIG. 4a-b, Table 6). The suboptimal PAMs with minimal cis-cleavage (K_{cleave} 0-0.1 min⁻¹) have worst performance in both collateral activity and one-pot reaction. In contrast, the suboptimal PAMs with intermediate K_{cleave} of 0.1-1.2 min⁻¹ outperformed canonical PAMs in one-pot reaction (FIG. 4a-b, Table 6). These results indicate that the efficiency of cis-cleavage is the key factor to determine the performance of one-pot reaction. Due to excessive substrates consumption caused by canonical PAMs-mediated cis-cleaving, the amplicon accumulation is slow and unstable, resulting in delayed or lack of collateral activity. Although suboptimal PAMs with minimal cis-cleavage allow accumulation of amplicon (substrates), they cannot execute sufficient collateral activity. In contrast, suboptimal PAMs with intermediate K_{cleave} allow substrates accumulation at the early stage of isothermal reaction, while maintain considerable collateral activity. We ranked the best performed suboptimal PAMs based on the time to half-maximum fluorescence in one-pot reaction (Table 6). In the top 5 best performed suboptimal PAMs for each spacer (20 PAMs in total), there are 12 VTTV and 5 TCTV. Therefore, we suggest that VTTV can be selected as the top selection of suboptimal PAMs, and TCTV are good candidates in the one-pot reaction.

Table 6, shown below, is a summary of ranked PAM by one-pot reaction performance and cis-cleavage activities. Ranking 120 PAMs by comparing performance in the one-pot reaction, “One pot reaction” represents time to half-maximum fluorescence (min)*an adjusted ratio based on plateau signal of each PAM in one-pot reaction, Kcleave represents cis-cleavage activities.

TABLE 6
One-pot reaction performance

| | HPV18 L1 gene | | | Orf1ab spacer 4 | | | Orf1ab spacer 5 | | | S gene spacer 2 | | |
|---|---|---|---|---|---|---|---|---|---|---|---|---|
| | | reaction | K_{cleave} | | reaction | K_{cleave} | | reaction | K_{cleave} | | reaction | K_{cleave} |
| No. | PAM | (min) | (min⁻¹) | PAM | (min) | (min⁻¹) | PAM | (min) | (min⁻¹) | PAM | (min) | (min⁻¹) |
| 1 | GTTA | 11.26 | 0.677 | TCTG | 9.75 | 0.905 | TTCA | 10.64 | 0.742 | ATTG | 14.44 | 0.295 |
| 2 | GTTG | 12.74 | 0.667 | TTAG | 10.19 | 0.540 | CTTA | 12.28 | 0.724 | TCTG | 15.23 | 0.409 |
| 3 | ATTA | 12.79 | 0.418 | GTTG | 10.44 | 0.675 | TIGA | 13.59 | 0.393 | TCTC | 15.87 | 0.451 |
| 4 | TCTA | 12.83 | 0.505 | TTGG | 11.28 | 0.534 | ATTA | 14.02 | 0.675 | TTAC | 16.98 | 0.198 |
| 5 | TIGG | 12.84 | 0.380 | TTAC | 11.47 | 0.594 | TATG | 14.79 | 0.410 | GTTC | 17.13 | 0.259 |
| 6 | TTGC | 12.98 | 0.499 | TTGC | 12.13 | 0.607 | GTTG | 14.88 | 0.180 | GTTG | 17.31 | 0.391 |
| 7 | TCTG | 13.33 | 0.207 | GTTC | 12.28 | 0.606 | CTTG | 14.93 | 0.856 | TTGC | 17.98 | 0.363 |
| 8 | ATTC | 13.33 | 0.306 | ATTC | 12.35 | 0.854 | TGTG | 14.97 | 0.476 | ATTC | 18.15 | 0.248 |
| 9 | GTTC | 13.47 | 8.329 | TCTC | 12.81 | 0.593 | ATTC | 15.21 | 0.126 | ATTA | 18.33 | 0.329 |
| 10 | TTCC | 13.58 | 0.486 | TCTA | 14.54 | 1.164 | GTTA | 15.21 | 0.589 | TTCA | 18.60 | 0.327 |
| 11 | TTGA | 13.59 | 0.354 | ATTG | 14.92 | 0.688 | TCTA | 15.30 | 0.682 | GTTA | 18.79 | 0.217 |
| 12 | TTCG | 13.60 | 1.164 | TTCG | 17.51 | 0.658 | CTTC | 15.56 | 0.154 | TTGG | 19.72 | 0.350 |
| 13 | TCTC | 13.70 | 0.474 | TTCA | 17.60 | 0.287 | TTGC | 15.86 | 0.163 | CITA | 19.74 | 0.341 |
| 14 | TTAC | 13.96 | 0.337 | CTTG | 17.75 | 0.943 | TTCC | 15.89 | 0.228 | TTAG | 21.37 | 0.334 |
| 15 | ATTG | 14.76 | 1.061 | TTCC | 18.07 | 0.643 | TCTC | 16.37 | 0.279 | TTGA | 21.38 | 0.164 |
| 16 | TTAG | 15.44 | 0.151 | TTGA | 19.67 | 1.102 | GTTC | 16.53 | 0.142 | TTCG | 21.49 | 0.417 |
| 17 | TTAA | 17.74 | 0.302 | ATTA | 22.85 | 1.162 | TATC | 16.57 | 0.121 | TTCC | 22.32 | 0.398 |
| 18 | TTCA | 17.80 | 0.526 | TATG | 23.01 | 0.105 | TTAC | 16.70 | 0.217 | CTTC | 25.11 | 0.290 |
| 19 | CTTC | 18.25 | 0.711 | TTAA | 24.79 | 0.371 | ATTG | 17.41 | 0.170 | TCTA | 25.91 | 0.386 |
| 20 | CITA | 22.53 | 0.548 | GTTA | 25.09 | 0.679 | TCTG | 18.19 | 0.619 | TTAA | 27.12 | 0.110 |
| 21 | CITG | 31.80 | 1.161 | CTTC | 25.44 | 0.925 | TTAA | 21.47 | 0.373 | CTTG | 28.62 | 0.224 |
| 22 | TATG | 32.20 | 0.114 | TTTG | 30.10 | 2.945 | TTCG | 21.65 | 0.656 | TTTA | 34.54 | 1.303 |
| 23 | TATA | 34.84 | 0.131 | TTTC | 34.60 | 3.192 | TTGG | 21.97 | 0.317 | TGTG | 34.62 | 0.054 |
| 24 | TTTC | 35.32 | 1.503 | TTTA | 45.02 | 3.395 | TTAG | 24.25 | 0.200 | TITG | 39.10 | 2.250 |
| 25 | TATC | 40.77 | 0.093 | CTTA | 49.47 | 1.755 | TTTC | 32.05 | 2.022 | TTTC | 40.45 | 1.276 |
| 26 | TTTA | 51.94 | 2.666 | TATA | 52.65 | 0.087 | TTTA | 37.02 | 1.725 | TGTC | 51.37 | 0.030 |
| 27 | TGTA | 66.13 | 0.086 | TATC | 54.29 | 0.101 | TTTG | 37.20 | 3.317 | TATG | 68.84 | 0.068 |
| 28 | TTTG | 71.77 | 2.675 | TGTG | 66.95 | 0.079 | TATA | 173.04 | 0.063 | TATA | 120.14 | 0.089 |
| 29 | TGTC | 92.56 | 0.077 | TGTC | 120.41 | 0.096 | TGTA | 185.69 | 0.098 | TATC | 168.58 | 0.058 |
| 30 | TGTG | 92.99 | 0.061 | TGTA | 143.56 | 0.098 | TGTC | 187.14 | 0.098 | TGTA | 205.49 | 0.075 |

Taken together, these data suggest a model for how suboptimal PAM functions to promote isothermal amplification and thus results in reliable and sensitive detection in a one-pot reaction (FIG. 4b). Given that CRISPR detection and isothermal amplification compete against each other in a one-pot reaction, the decreased binding affinity of Cas12a for suboptimal PAM substrates promotes a shift of the balance from cleavage towards amplification and thus generates sufficient amplicons for detection; in contrast, the stronger binding affinity of canonical PAM allows cleavage to outcompete amplification and leads to delay or lack of amplicon production, which is responsible for the observed delay and instability of detection.

We have demonstrated that using suboptimal PAM is superior for one-pot reaction. Those results were obtained using a RPA kit (AMP future). To examine whether this conclusion is valid using a different RPA kit, we performed experiments with a RPA kit from TwistDx. These two RPA kits showed no difference in amplification sensitivity (FIG. 19a). However, when we combined RPA and Cas12a in one reaction, TwistDx kit showed much less sensitivity and reduced fluorescence signal (FIG. 19b). A small volume of Cas12a RNP (2 μL) was added into RPA (18 μL) for one-pot reaction. It suggests that Cas12a is not fully compatible with buffer environment of TwistDx RPA. A recent study showed that LwaCas13a was also not fully compatible with TwistDx RPA buffer environment. Arizti-Sanz et al. optimized buffer, making it suitable for both RPA amplification and Cas13a activity, a method named SHINE. Inspired by this study, we increased RNP doses from 33.3 nM to 100 nM, 200 nM and 333 nM in the reaction. We found 3-6 folds access of RNP can substantially improve the fluorescence curve of one-pot reaction (FIG. 19c). Using this improved condition and TwistDx kit, we compared the performance of canonical PAMs and suboptimal PAMs of four spacers. Similar to AMP future kits, the suboptimal PAMs perform much better than canonical PAMs using TwistDx kit (FIG. 19d-g).

We have thus developed the Flexible, Accelerated, Suboptimal PAM-based Test with Enhanced sensitivity and Reproducibility (FASTER). To determine whether FASTER is able to detect DNA viruses, we tested the method with human cytomegalovirus (HCMV), a double-stranded DNA virus. We first used a plasmid containing the UL55 gene sequence of HCMV as a substrate to compare the sensitivity of FASTER with qPCR. Both assays showed the same detection limit, 5.953×10⁻⁴ amol plasmid per reaction (equal to 29.765 aM concentration in qPCR and 19.843 aM in FASTER) (FIG. 5a-b). Strikingly, the fluorescence signal of FASTER started to appear at approximately 6-10 minutes and reached a half-maximum at approximately 9-15 minutes for all concentrations tested (FIG. 5b). Notably, this speed is at least 2 to 3-fold faster than that of all published CRISPR-mediated one-pot tests with target amplification. We then used FASTER to measure the HCMV viral samples. The results showed a detection limit of 24 copies per reaction, comparable to that of qPCR (FIG. 5c-d). To enable a broader application of FASTER, we used a simple UV light instead of fluorescence spectroscopy to measure the signal. At the 10-minute time point, all except the lowest viral concentration showed positive signals on UV detection, and at 15 minutes, the sample with the lowest number of viral copies (equal to a qPCR Ct value of 36) was clearly positive (FIG. 5e-f). We also combined FASTER with lateral-flow assay strips, and this combination was able to detect viral samples with Ct values of 33-34 (FIG. 5g). These results are in agreement with previous studies showing that lateral-flow strip assays are less sensitive than their corresponding fluorescence signal-based assays. The readout by lateral flow requires opening of tubes to add buffer and a strip, an additional step which increases hands-on and waiting times, as well as risk of cross-contamination. As fluorescence readout stimulated by a simple UV light is simpler, faster and more sensitive, we decided to use it for FASTER to detect SARS-CoV-2 samples.

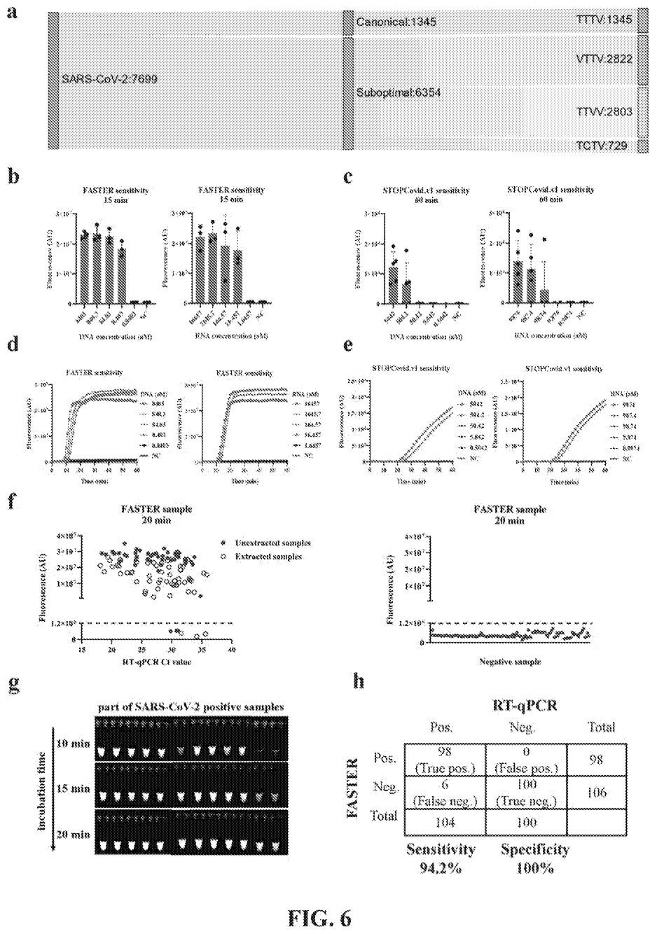

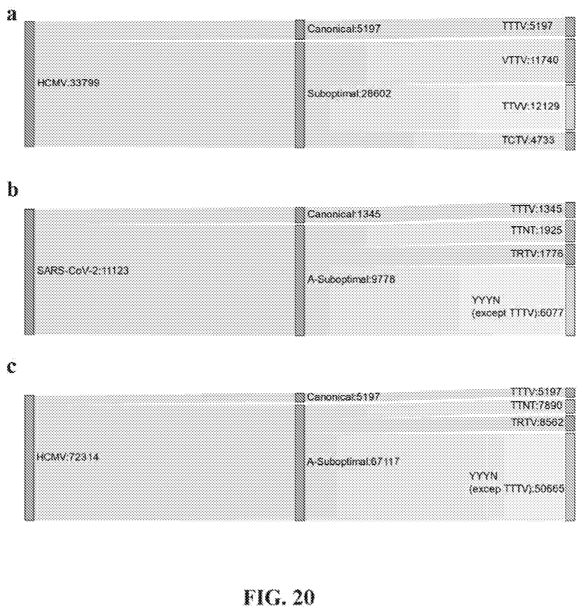

One strength of FASTER is that it greatly expands the available selection of crRNAs as there are more suboptimal PAMs than canonical PAMs. Spacers using VTTV, TCTV and TTVV PAMs likely perform well in the one-pot reaction, making the number of available suboptimal PAMs 7-fold higher than that of canonical PAMs in theory (21 combinations vs 3 combinations) (FIG. 6a, FIG. 20a). Moreover, some additional suboptimal PAMs, such as TRTV, TTNT and YYYN (except TTTV) may also function better than canonical PAMs, making the choice of spacer even more flexible (FIG. 20b-c). The relaxed criteria of PAM selection are particularly important for developing test kits for viral detection. Although there are more than 1000 canonical PAMs of Cas12a in SARS-CoV-2, only a limited number of canonical PAMs could be employed for viral detection assays given the selection criteria: 1) in a conserved region; 2) in a high-copy gene; 3) an active crRNA; 4) compatible with robust primers for isothermal amplification. Hence, the extended selection of suboptimal PAMs makes FASTER more flexible for assay optimization and application to new viral strains.

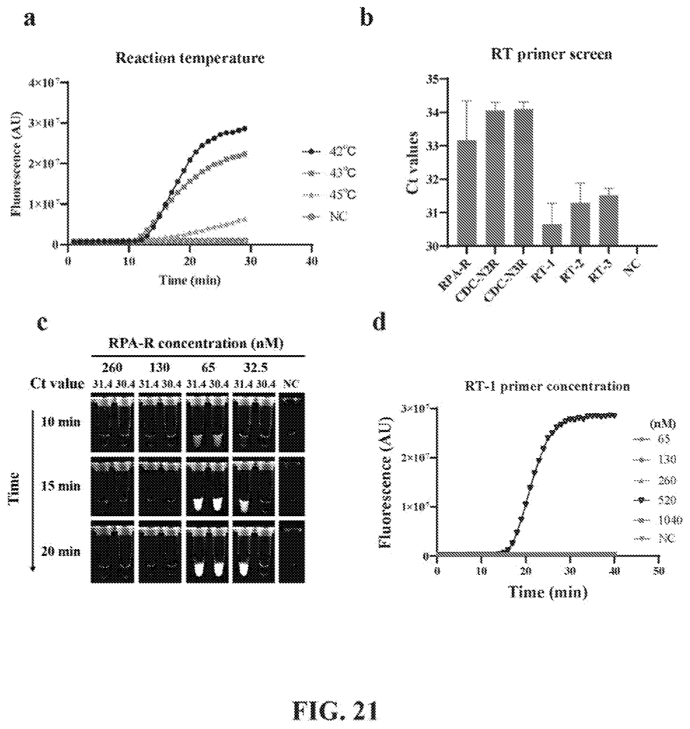

Finally, we applied FASTER to detect SARS-CoV-2. We first compared the sensitivity of RPA/Cas12a/suboptimal PAM-based FASTER and LAMP/Cas12b-based STOPCovid using a DNA fragment encoded N gene of SARS-CoV-2. FASTER is ˜100-fold more sensitive than STOPCovid, and the speed of FASTER is 3-fold faster than STOPCovid in detecting DNA samples (13 minutes vs 40 minutes, time to half-maximum fluorescence) (FIG. 6b-e). Then we examined the capability of FASTER to detect RNA samples by combining RT-RPA and Cas12a. Our initial data showed that the FASTER was not as sensitive as RT-qPCR to detect RNA samples. We speculated the RT step was the rate-limiting step, despite RNase H has been added to the reaction. To improve the efficiency of RT step, we first increase the temperature of reaction from 37° C. to 42° C., as RT enzyme usually perform better with higher temperature, and both RPA and Cas12a are activate at 42° C. Indeed, FASTER performed well at 42° C. (FIG. 21a). Similar to RT-qPCR, RT-RPA usually used its reverse primer as RT primer. We hypothesized that the RPA reverse primer which is more than 30 nt, may not be efficient for RT step. Indeed, the RT efficiency of RPA reverse primer was 6-fold less efficient than qPCR reverse primer (FIG. 21b). Therefore, we added an additional 18 nt primer to function as a RT primer, and meanwhile reduce the concentration of RPA reverse primer to prevent its inference of RT process. The combination of reactions at 42° C., adding an additional short RT primer and reducing the concentration of RPA reverse primer significantly improved RT efficiency and overall FASTER performance for detecting RNA samples (FIG. 21a-d). An in vitro-transcribed RNA fragment of SARS-CoV-2 N gene was used to compare the ability of FASTER and STOPCovid to detect RNA. FASTER exhibited ˜100-fold higher sensitivity, and ˜2.5-fold faster than STOPCovid for RNA detection (FIG. 6b-e).

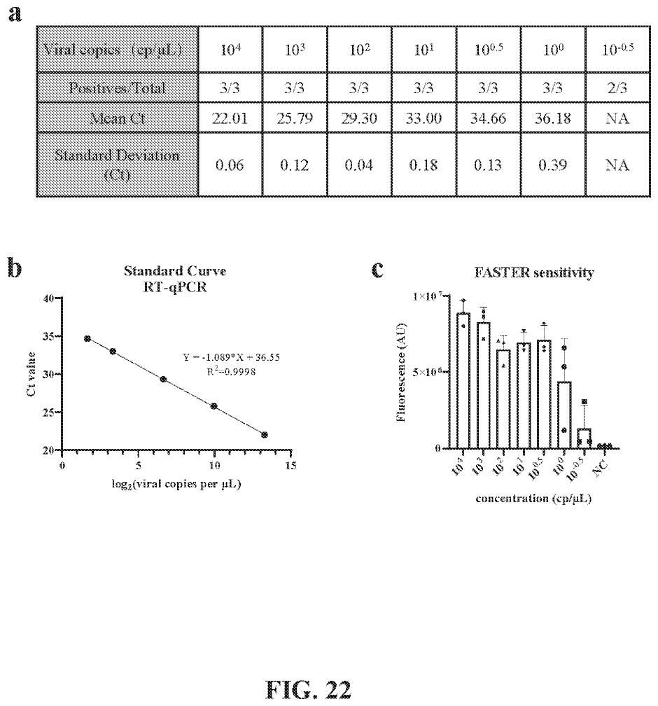

We head-to-head compared limit of detection (LOD) of FASTER and RT-qPCR, the latter known as current golden standard by US Centers for Disease Control and Prevention (CDC). We first determined the LOD of RT-qPCR using commercial available SARS-CoV-2 Pseudovirus as the standard. The LOD of RT-qPCR is 1 cp/μL (FIG. 22a-b), which is consistent with the results released by CDC. Then we compared LOD of FASTER and RT-qPCR, and identified that the LOD of FASTER is 1 cp/μL (FIG. 22c), comparable to that of RT-qPCR.

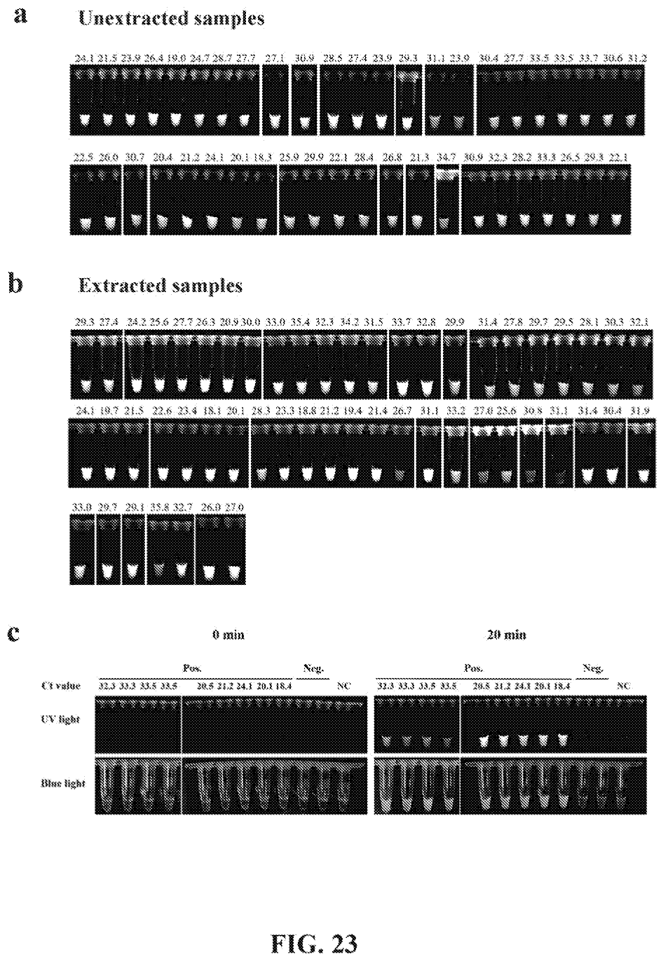

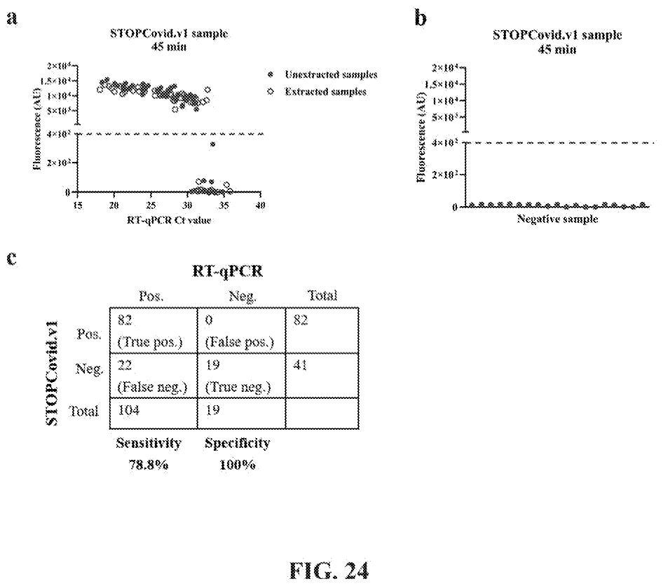

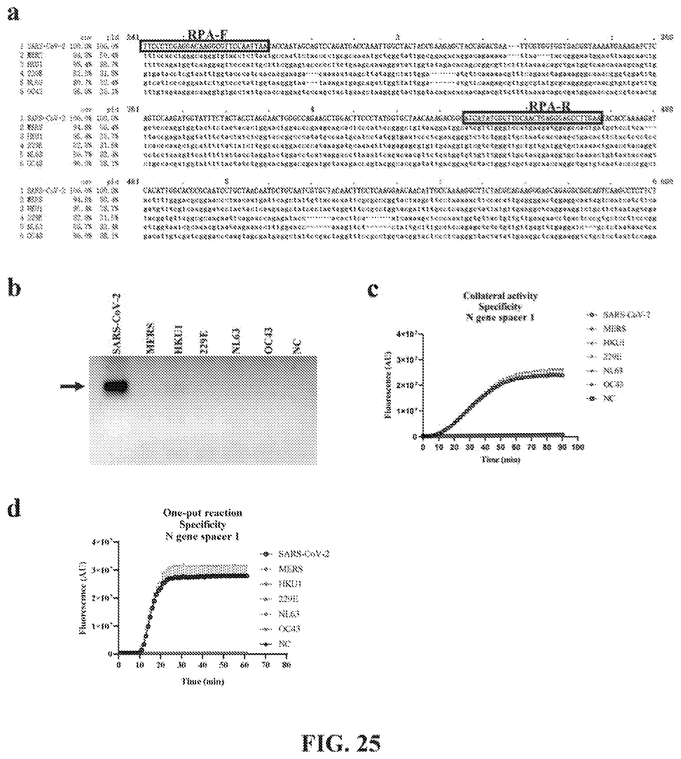

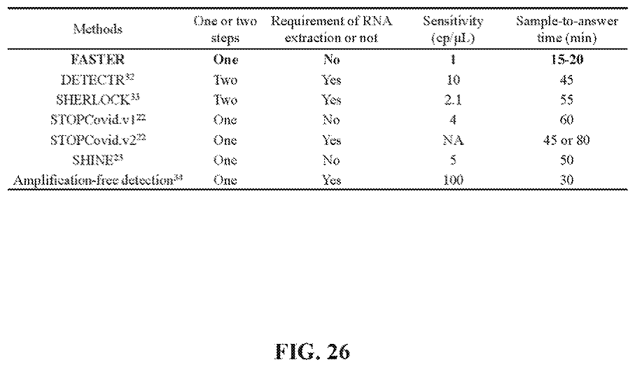

We evaluated the performance of FASTER in SARS-CoV-2-positive and -negative clinical samples, and compared its performance with STOPCovid. A total of 104 SARS-CoV-2-positive nasopharyngeal swab samples (48 unextracted and 56 extracted samples) with a wide range of Ct values (from 18.1 to 35.8) and 100 SARS-CoV-2-negative samples were used. FASTER had a sensitivity of 94.2% and a specificity of 100.0%, and it was able to detect samples with Ct value 35.8 (1.2 cp/μL according to standard curve of RT-qPCR in FIG. 22b) (FIG. 6f-h). The positive signal appeared as early as 10 minutes, and all positive samples showed signals at 15 minutes (FIG. 6h, FIG. 23a). The signal can be detected by UV light or a simple blue light device (FIG. 23b). In comparison, STOPCovid.v1 was unable to stably detect samples with Ct value above 31.0, resulting a sensitivity of 78.8% (FIG. 24a-c). Finally, to assess the specificity of FASTER, we tested several common human coronaviruses including MERS, HKU1, 229E, NL63 and OC43 through RPA amplification, collateral activity test and one-pot reaction. These results indicate no cross-react with other common viruses (FIG. 25a-d).

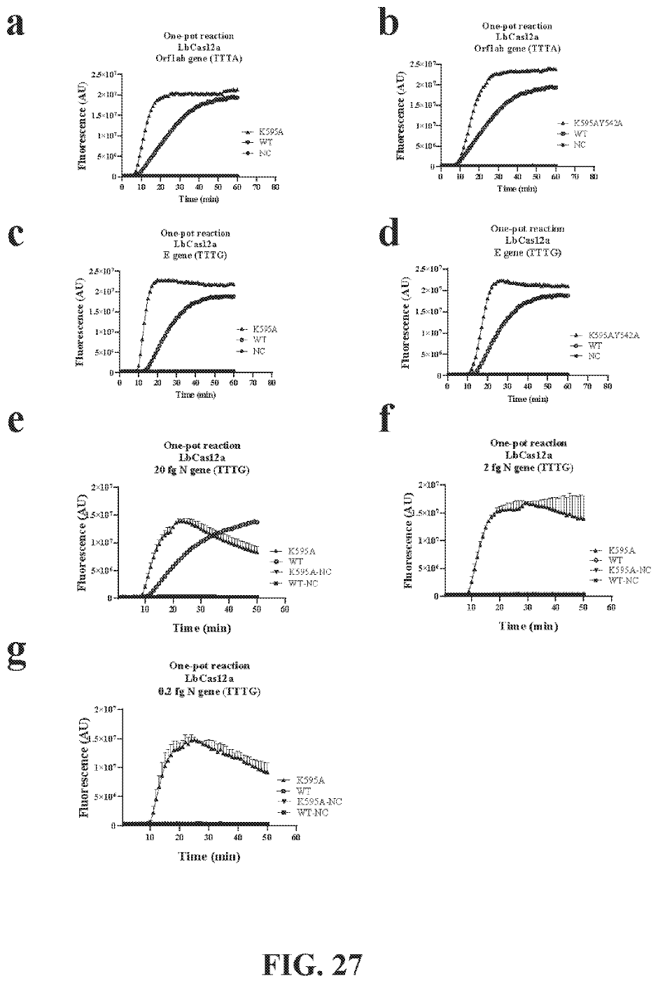

Using suboptimal PAM for one-pot test could be applied for other members of Cas12a family and effectors of Class II type V. It will be interesting to explore whether other Cas proteins could exhibit superior speed using suboptimal PAM in one-pot reaction than Cas12a. Hence, we also provide an alternative rapid and sensitive detection method using cas protein mutants. We mutated the amino acid that forms hydrogen bonds with PAM on the LbCas12a protein to alanine, and then used the mutant protein to target the canonical PAM to establish a rapid one-step detection. As we expected, the two mutants of K595A and K595A&Y542A reached the plateau phase faster than the wild-type protein in the one-step reaction, especially K595A can reach the peak within 20 minutes (FIG. 27a-d). In addition, the sensitivity of the mutant is also increased by 100 times compared to the wild type. The limit of detection of K595A mutant is 16.457 aM N gene dsDNA, while wild type could only identify 1645.7 aM dsDNA (FIG. 27e-g).

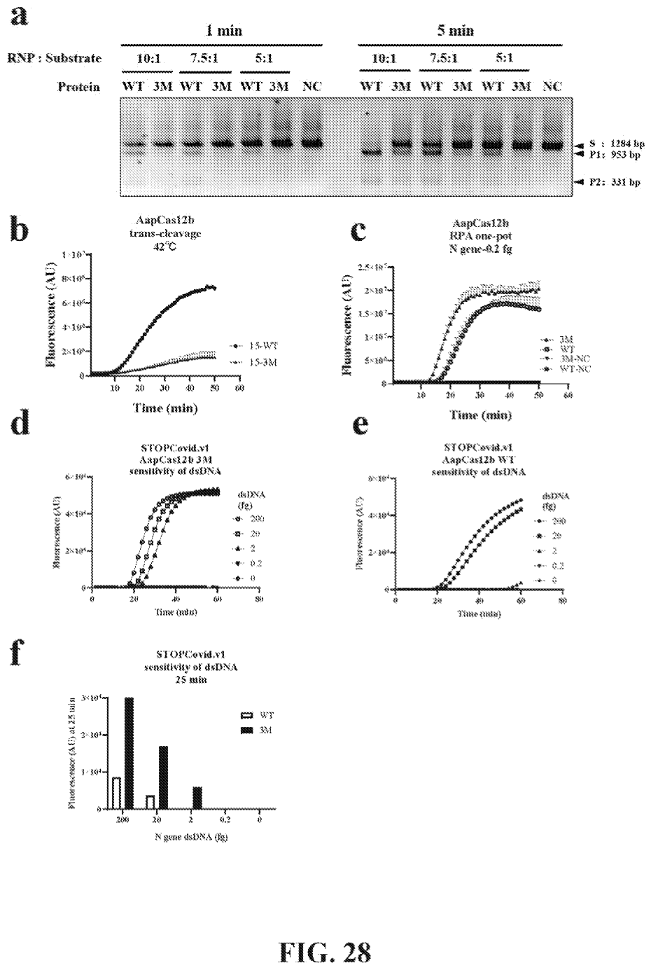

The one-step method of cas12a and RPA is to react at 37-42° C. The reverse transcription step is included for detection of RNA virus samples, and higher temperatures may be beneficial for this step. High-temperature resistant such as Cas12b can be combined with high-temperature isothermal amplification methods such as LAMP. We mutated the residues 478G, 396K and 403Q associated PAM into alanine for Cas12b, here abbreviated the mutant as 3M (3 point mutations together). The results of cis-cleavage and trans-cleavage indicate that the activity of 3M is indeed weaker than that of WT (FIG. 28a-b). In the RPA-mediated one-step method, the reaction speed of 3M is significantly faster than that of WT (FIG. 28c). Next, we tested it in the LAMP-mediated one-pot reaction, and the results were consistent with the RPA-mediated one-pot reaction. Not only the reaction speed was accelerated, but the sensitivity was increased by 10 times (FIG. 28d-e). In short, with 3M Cas12b protein, 164.57 aM samples can be clearly detected within 25 minutes (FIG. 28f).

Taken together, the FASTER detection developed in our study showed less incubation time than STOP, and more sensitive than Cas13a-based detection without pre-amplification. FASTER detection is the first CRISPR-mediated detection with the following characteristics in combination: fast speed, high sensitivity, high reliability and flexibility.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for detecting a target polynucleotide, comprising incubating the target polynucleotide in a mixture that comprises (a) a polymerase, (b) deoxynucleoside triphosphates (dNTPs), (c) primers for amplifying the target polynucleotide, (c) a CRISPR-associated (Cas) nuclease, and (d) a guide RNA comprising a spacer fragment complementary to a target fragment on the target polynucleotide, under conditions so that the polymerase effectively amplifies the target polynucleotide while the Cas nuclease is capable of cleaving the amplified target polynucleotide.

2. The method of claim 1, wherein the target fragment for the guide RNA includes, or is adjacent to, a protospacer adjacent motif (PAM) sequence recognizable by the Cas nuclease, which PAM sequence is suboptimal.

3. The method of claim 2, wherein the PAM sequence is not canonical.

4. The method of claim 2, wherein the Cas nuclease is LbCas12a, and the PAM sequence is selected from the group consisting of NTTV, TNTV, TTNV, TTNT, VTTT, TVTT, VVTT, VTVT, VNVV, NVNV, NVVV, VNTV, NTVV, TNVV, YYYN and VVNV, wherein N denotes any nucleotide.

5. The method of claim 2, wherein the Cas nuclease is AapCas12b and the PAM sequence is selected from the group consisting of VTN, TTN, TVN, NVN and VVN, wherein N denotes any nucleotide.

6. The method of claim 1, wherein at least one of the dNTPs is modified.

7. The method of claim 6, wherein the modification is with a group selected from the group consisting of phosphoryl, biotin, digoxigenin, amino, thiol, phosphorthioate, and methyl.

8-10. (canceled)

11. The method of claim 1, wherein the guide RNA comprises a truncated or 5′/3′ DNA-/RNA-extended CRISPR RNA (crRNA) that includes the spacer fragment.

12-13. (canceled)

14. The method of claim 1, wherein the guide RNA includes a truncated trans-activating crispr RNA (tracrRNA) sequence.

15. (canceled)

16. The method of claim 1, wherein at least one of the nucleotides in the spacer fragment is a not a standard ribonucleotide.

17. The method of claim 16, wherein at least one of the nucleotides in the spacer fragment is selected from the group consisting of a deoxynucleotide, a locked nucleic acid (LNA), a bridged nucleic acid (BNA), a deoxyuridine, a deoxyinosine, a pseudouridin, a methylpseudouridin, and modified nucleotide, wherein the modification is with a group selected from the group consisting of phosphoryl, biotin, digoxigenin, amino, thiol, phosphorthioate, methyl, 2′-O-methyl-3′-phosphonoacetate (MP), 2′O-methoxyethyl (MOE), Fluoro(F), S-constrained ethyl, 2′-O-methyl-PS (MS) and 2′-O-methyl-thioPACE (MSP).

18-20. (canceled)

21. The method of claim 1, wherein the Cas nuclease is sequence engineered.

22. The method of claim 21, wherein the sequence engineering changes the activity of the Cas nuclease in forming a ribonucleoprotein (RNP) or binding substrate nucleic acid.

23. The method of claim 22, wherein the sequence engineered Cas nuclease is LbCas12a with one or more amino acid deletion or substitution at a residue selected from the group consisting of Lys15, Thr16, Arg18, Lys20, Lys51, Asn157, Arg158, Arg174, Lys253, Gln264, Lys278, Leu281, Arg386, Lys390, Lys464, Arg508, Lys520, Lys707, Ser710, Thr713, His714, Gly715, Thr716, Asn718, His720, Arg747, Ala766, Asn767, Lys768, Asn769, Asn772, Lys774, Thr777, Tyr781, Asp786, Arg788, Gln793, Asn808, Tyr872, Glu898, Lys953, and Lys960.

24-27. (canceled)

28. The method of claim 21, wherein the sequence engineering changes the conformation of the Cas nuclease.

29. The method of claim 28, wherein the sequence engineered Cas nuclease is LbCas12a with one or more amino acid deletion or substitution at a residue selected from the group consisting of Lys457, Val511, Thr512, Gln888 and Try890, or is LbuCas13a with one or more amino acid deletion or substitution at a residue selected from the group consisting of Lys2, Lys5, Gln371, Phe375, Lys783 and His962.

30. The method of claim 21, wherein the sequence engineering changes the activity of the Cas nuclease in interacting with a PAM sequence.

31. The method of claim 30, wherein the sequence engineered Cas nuclease is LbCas12a with one or more amino acid deletion or substitution at a residue selected from the group consisting of Lys121, Thr148, Thr149, Trp534, Asp535, Lys538, Tyr542, Lys595, Ser599, Lys600, Lys601, Try616, Try646, Trp649, and Gly740, or is AapCas12b with one or more amino acid deletion or substitution at a residue selected from the group consisting of Lys209, Lys141, Ala142, Lys145, Asn144, Gly143, Asn400, Lys396, Gln118, Gly478, Arg507, Gln403, Arg208, Gln211, Ala212, Val213, Arg218, Val134, Gly135, Leu137, Gly136, Gln119, Arg122, Gly143, Asn144, and Arg150, Arg147.

32-44. (canceled)

45. A kit or package for detecting a target polynucleotide, comprising (a) a polymerase, (b) deoxynucleoside triphosphates (dNTPs), (c) primers for amplifying the target polynucleotide, (c) a CRISPR-associated (Cas) nuclease, and (d) a guide RNA comprising a spacer fragment complementary to a target fragment on the target polynucleotide, wherein the polymerase can effectively amplify the target polynucleotide while the Cas nuclease is capable of cleaving the amplified target polynucleotide.

46-47. (canceled)

48. A mutant Cas nuclease having (a) reduced activity in forming a ribonucleoprotein (RNP), (b) changed conformation, (c) reduced activity in interacting with a target PAM sequence, or (d) reduced binding to a target polynucleotide to be cleaved.


SEQUENCE LISTING (109 sequences)

SEQ ID NO: 1 - DNA, 31 bp, Artificial Sequence
  Synthetic
ctaaagcttacaaagattatctagctagtgg

SEQ ID NO: 2 - DNA, 33 bp, Artificial Sequence
  Synthetic
tttgtacatacttaccttttaagtcacaaaatc

SEQ ID NO: 3 - DNA, 31 bp, Artificial Sequence; identical to SEQ ID NO: 1
  Synthetic

SEQ ID NO: 4 - DNA, 33 bp, Artificial Sequence; identical to SEQ ID NO: 2
  Synthetic

SEQ ID NO: 5 - DNA, 30 bp, Artificial Sequence
  Synthetic
aggtttcaaactttacttgctttacataga

SEQ ID NO: 6 - DNA, 30 bp, Artificial Sequence
  Synthetic
tcctaggttgaagataacccacataataag

SEQ ID NO: 7 - DNA, 30 bp, Artificial Sequence; identical to SEQ ID NO: 5
  Synthetic

SEQ ID NO: 8 - DNA, 30 bp, Artificial Sequence; identical to SEQ ID NO: 6
  Synthetic

SEQ ID NO: 9 - DNA, 30 bp, Artificial Sequence; identical to SEQ ID NO: 5
  Synthetic

SEQ ID NO: 10 - DNA, 31 bp, Artificial Sequence
  Synthetic
gcatctgtaatggttccattttcattatatt

SEQ ID NO: 11 - DNA, 31 bp, Artificial Sequence
  Synthetic
atgtactcattcgtttcggaagagacaggta

SEQ ID NO: 12 - DNA, 33 bp, Artificial Sequence
  Synthetic
ttagaccagaagatcaggaactctagaagaatt

SEQ ID NO: 13 - DNA, 32 bp, Artificial Sequence
  Synthetic
atgattatgtgactcgcacaagcatattttat

SEQ ID NO: 14 - DNA, 32 bp, Artificial Sequence
  Synthetic
actaaacgttgtgtttcaggattataaatact

SEQ ID NO: 15 - DNA, 29 bp, Artificial Sequence
  Synthetic
tagctacgcttacatctacaccacttatc

SEQ ID NO: 16 - DNA, 33 bp, Artificial Sequence
  Synthetic
taggaactgtagcattgagcaaacttgttgatg

SEQ ID NO: 17 - DNA, 29 bp, Artificial Sequence
  Synthetic
ttccctcgaggacaaggcgttccaattaa

SEQ ID NO: 18 - DNA, 32 bp, Artificial Sequence
  Synthetic
ttcaaggctccctcagttgcaacccatatgat

SEQ ID NO: 19 - RNA, 41 nt, Artificial Sequence
  Synthetic
uaauuucuacuaaguguagauccacauagaucauccaaauc

SEQ ID NO: 20 - RNA, 41 nt, Artificial Sequence
  Synthetic
uaauuucuacuaaguguagauacacaauuagugauugguug

SEQ ID NO: 21 - RNA, 41 nt, Artificial Sequence
  Synthetic
uaauuucuacuaaguguagaugacagcuggugcugcagcuu

SEQ ID NO: 22 - RNA, 41 nt, Artificial Sequence
  Synthetic
uaauuucuacuaaguguagauucagguuggacagcuggugc

SEQ ID NO: 23 - RNA, 41 nt, Artificial Sequence
  Synthetic
uaauuucuacuaaguguagauuguggguuaucuucaaccua

SEQ ID NO: 24 - RNA, 41 nt, Artificial Sequence
  Synthetic
uaauuucuacuaaguguagauauugugugcguacugcugca

SEQ ID NO: 25 - RNA, 41 nt, Artificial Sequence
  Synthetic
uaauuucuacuaaguguagaucaacaguuaauaaucuagag

SEQ ID NO: 26 - RNA, 41 nt, Artificial Sequence
  Synthetic
uaauuucuacuaaguguagauugugaugaaucucccacaua

SEQ ID NO: 27 - RNA, 41 nt, Artificial Sequence
  Synthetic
uaauuucuacuaaguguagauucaucuggacugcuauuggu

SEQ ID NO: 28 - DNA, 24 bp, Artificial Sequence
  Synthetic
gttgccacatagatcatccaaatc

SEQ ID NO: 29 - DNA, 24 bp, Artificial Sequence
  Synthetic
caacggtgtatctagtaggtttag

SEQ ID NO: 30 - DNA, 24 bp, Artificial Sequence
  Synthetic
cttaacacaattagtgattggttg

SEQ ID NO: 31 - DNA, 24 bp, Artificial Sequence
  Synthetic
gaattgtgttaatcactaaccaac

SEQ ID NO: 32 - DNA, 24 bp, Artificial Sequence
  Synthetic
gttggacagctggtgctgcagctt

SEQ ID NO: 33 - DNA, 24 bp, Artificial Sequence
  Synthetic
caacctgtcgaccacgacgtcgaa

SEQ ID NO: 34 - DNA, 24 bp, Artificial Sequence
  Synthetic
ttcttcaggttggacagctggtgc

SEQ ID NO: 35 - DNA, 24 bp, Artificial Sequence
  Synthetic
aagaagtccaacctgtcgaccacg

SEQ ID NO: 36 - DNA, 24 bp, Artificial Sequence
  Synthetic
attatgtgggttatcttcaaccta

SEQ ID NO: 37 - DNA, 24 bp, Artificial Sequence
  Synthetic
taatacacccaatagaagttggat

SEQ ID NO: 38 - DNA, 24 bp, Artificial Sequence
  Synthetic
ttcgattgtgtgcgtactgctgca

SEQ ID NO: 39 - DNA, 24 bp, Artificial Sequence
  Synthetic
aagctaacacacgcatgacgacgt

SEQ ID NO: 40 - DNA, 24 bp, Artificial Sequence
  Synthetic
ttaccaacagttaataatctagag

SEQ ID NO: 41 - DNA, 24 bp, Artificial Sequence
  Synthetic
aatggttgtcaattattagatctc

SEQ ID NO: 42 - DNA, 24 bp, Artificial Sequence
  Synthetic
ttgatgtgatgaatctcccacata

SEQ ID NO: 43 - DNA, 24 bp, Artificial Sequence
  Synthetic
aactacactacttagagggtgtat

SEQ ID NO: 44 - DNA, 24 bp, Artificial Sequence
  Synthetic
ttggtcatctggactgctattggt

SEQ ID NO: 45 - DNA, 24 bp, Artificial Sequence
  Synthetic
aaccagtagacctgacgataacca

SEQ ID NO: 46 - DNA, 43 bp, Artificial Sequence
  Synthetic
ggagtgagtacggtgtgcaatttctaaagcttacaaagattat

SEQ ID NO: 47 - DNA, 43 bp, Artificial Sequence
  Synthetic
ggagtgagtacggtgtgcctattctaaagcttacaaagattat

SEQ ID NO: 48 - DNA, 43 bp, Artificial Sequence
  Synthetic
ggagtgagtacggtgtgccgattctaaagcttacaaagattat

SEQ ID NO: 49 - DNA, 43 bp, Artificial Sequence
  Synthetic
ggagtgagtacggtgtgcaggcgctaaagcttacaaagattat

SEQ ID NO: 50 - DNA, 43 bp, Artificial Sequence
  Synthetic
ggagtgagtacggtgtgctcctcctaaagcttacaaagattat

SEQ ID NO: 51 - DNA, 43 bp, Artificial Sequence
  Synthetic
ggagtgagtacggtgtgcactaactaaagcttacaaagattat

SEQ ID NO: 52 - DNA, 43 bp, Artificial Sequence
  Synthetic
ggagtgagtacggtgtgcgtggcctaaagcttacaaagattat

SEQ ID NO: 53 - DNA, 43 bp, Artificial Sequence
  Synthetic
ggagtgagtacggtgtgcataaactaaagcttacaaagattat

SEQ ID NO: 54 - DNA, 43 bp, Artificial Sequence
  Synthetic
ggagtgagtacggtgtgccacgcctaaagcttacaaagattat

SEQ ID NO: 55 - DNA, 43 bp, Artificial Sequence
  Synthetic
ggagtgagtacggtgtgcgtagcctaaagcttacaaagattat

SEQ ID NO: 56 - DNA, 43 bp, Artificial Sequence
  Synthetic
ggagtgagtacggtgtgcgaagtctaaagcttacaaagattat

SEQ ID NO: 57 - DNA, 43 bp, Artificial Sequence
  Synthetic
ggagtgagtacggtgtgcctgtgctaaagcttacaaagattat

SEQ ID NO: 58 - DNA, 43 bp, Artificial Sequence
  Synthetic
ggagtgagtacggtgtgcacccactaaagcttacaaagattat

SEQ ID NO: 59 - DNA, 43 bp, Artificial Sequence
  Synthetic
ggagtgagtacggtgtgcgggtgctaaagcttacaaagattat

SEQ ID NO: 60 - DNA, 43 bp, Artificial Sequence
  Synthetic
ggagtgagtacggtgtgcgagatctaaagcttacaaagattat

SEQ ID NO: 61 - DNA, 43 bp, Artificial Sequence
  Synthetic
ggagtgagtacggtgtgcgcgcgctaaagcttacaaagattat

SEQ ID NO: 62 - DNA, 43 bp, Artificial Sequence
  Synthetic
gagttggatgctggatggacaagtttgtacatacttacctttt

SEQ ID NO: 63 - DNA, 43 bp, Artificial Sequence
  Synthetic
gagttggatgctggatggtggcttttgtacatacttacctttt

SEQ ID NO: 64 - DNA, 43 bp, Artificial Sequence
  Synthetic
gagttggatgctggatggtgccctttgtacatacttacctttt

SEQ ID NO: 65 - DNA, 43 bp, Artificial Sequence
  Synthetic
gagttggatgctggatggatttctttgtacatacttacctttt

SEQ ID NO: 66 - DNA, 43 bp, Artificial Sequence
  Synthetic
gagttggatgctggatggccctgtttgtacatacttacctttt

SEQ ID NO: 67 - DNA, 43 bp, Artificial Sequence
  Synthetic
gagttggatgctggatggtcatttttgtacatacttacctttt

SEQ ID NO: 68 - DNA, 43 bp, Artificial Sequence
  Synthetic
gagttggatgctggatgggcgtatttgtacatacttacctttt

SEQ ID NO: 69 - DNA, 43 bp, Artificial Sequence
  Synthetic
gagttggatgctggatggccgcatttgtacatacttacctttt

SEQ ID NO: 70 - DNA, 43 bp, Artificial Sequence
  Synthetic
gagttggatgctggatggacacgtttgtacatacttacctttt

SEQ ID NO: 71 - DNA, 43 bp, Artificial Sequence
  Synthetic
gagttggatgctggatggcttcgtttgtacatacttacctttt

SEQ ID NO: 72 - DNA, 43 bp, Artificial Sequence
  Synthetic
gagttggatgctggatgggagcgtttgtacatacttacctttt

SEQ ID NO: 73 - DNA, 43 bp, Artificial Sequence
  Synthetic
gagttggatgctggatgggtacgtttgtacatacttacctttt

SEQ ID NO: 74 - DNA, 43 bp, Artificial Sequence
  Synthetic
gagttggatgctggatgggtttatttgtacatacttacctttt

SEQ ID NO: 75 - DNA, 43 bp, Artificial Sequence
  Synthetic
gagttggatgctggatggcgctctttgtacatacttacctttt

SEQ ID NO: 76 - DNA, 43 bp, Artificial Sequence
  Synthetic
gagttggatgctggatggttgaatttgtacatacttacctttt

SEQ ID NO: 77 - DNA, 43 bp, Artificial Sequence
  Synthetic
gagttggatgctggatgggatcgtttgtacatacttacctttt

SEQ ID NO: 78 - DNA, 23 bp, Artificial Sequence
  Synthetic
ctaccctcaagtacggagatgtg

SEQ ID NO: 79 - DNA, 23 bp, Artificial Sequence
  Synthetic
gtcttcattgataggcttcatcg

SEQ ID NO: 80 - DNA, 28 bp, Artificial Sequence
  Synthetic
atcttattcgctttgaacgtaatatcat

SEQ ID NO: 81 - DNA, 20 bp, Artificial Sequence
  Synthetic
ttacaaacattggccgcaaa

SEQ ID NO: 82 - DNA, 18 bp, Artificial Sequence
  Synthetic
gcgcgacattccgaagaa

SEQ ID NO: 83 - DNA, 21 bp, Artificial Sequence
  Synthetic
tggaaccaccttgtaggtttg

SEQ ID NO: 84 - DNA, 20 bp, Artificial Sequence
  Synthetic
tatgcaccaccgggtaaagt

SEQ ID NO: 85 - DNA, 21 bp, Artificial Sequence
  Synthetic
tggaaccaccttgtaggtttg

SEQ ID NO: 86 - DNA, 20 bp, Artificial Sequence
  Synthetic
acccacagggtcattagcac

SEQ ID NO: 87 - DNA, 21 bp, Artificial Sequence
  Synthetic
tggaaccaccttgtaggtttg

SEQ ID NO: 88 - DNA, 20 bp, Artificial Sequence
  Synthetic
acccacagggtcattagcac

SEQ ID NO: 89 - DNA, 23 bp, Artificial Sequence
  Synthetic
acacattattatttgtggccatt

SEQ ID NO: 90 - DNA, 20 bp, Artificial Sequence
  Synthetic
gtgcctttagcccagtgttc

SEQ ID NO: 91 - DNA, 21 bp, Artificial Sequence
  Synthetic
tggaaccaccttgtaggtttg

SEQ ID NO: 92 - DNA, 22 bp, Artificial Sequence
  Synthetic
ttcgcggagttgatcacaacta

SEQ ID NO: 93 - DNA, 22 bp, Artificial Sequence
  Synthetic
ttggatggaaagtgagttcaga

SEQ ID NO: 94 - DNA, 20 bp, Artificial Sequence
  Synthetic
acttcaccaaaagggcacaa

SEQ ID NO: 95 - DNA, 18 bp, Artificial Sequence
  Synthetic
atgtgatcttttggtgta

SEQ ID NO: 96 - DNA, 18 bp, Artificial Sequence
  Synthetic
atcttttggtgtattcaa

SEQ ID NO: 97 - DNA, 17 bp, Artificial Sequence
  Synthetic
ttgcagcattgttagca

SEQ ID NO: 98 - DNA, 23 bp, Artificial Sequence
  Synthetic
ggatttggatgatctatgtggca

SEQ ID NO: 99 - DNA, 19 bp, Artificial Sequence
  Synthetic
gtggtgcatcgtgttgtct

SEQ ID NO: 100 - DNA, 20 bp, Artificial Sequence
  Synthetic
gatgatctatgtggcaacgg

SEQ ID NO: 101 - DNA, 20 bp, Artificial Sequence
  Synthetic
ccacatagatcatccaaatc

SEQ ID NO: 102 - DNA, 20 bp, Artificial Sequence
  Synthetic
acacaattagtgattggttg

SEQ ID NO: 103 - DNA, 20 bp, Artificial Sequence
  Synthetic
taacgtgagtcttgtaaaac

SEQ ID NO: 104 - DNA, 20 bp, Artificial Sequence
  Synthetic
ttgctttcgtggtattcttg

SEQ ID NO: 105 - DNA, 20 bp, Artificial Sequence
  Synthetic
gtggtattcttgctagttac

SEQ ID NO: 106 - DNA, 20 bp, Artificial Sequence
  Synthetic
caagactcacgttaacaata

SEQ ID NO: 107 - DNA, 20 bp, Artificial Sequence
  Synthetic
cgtttactctcgtgttaaaa

SEQ ID NO: 108 - DNA, 20 bp, Artificial Sequence
  Synthetic
ctctcgtgttaaaaatctga

SEQ ID NO: 109 - DNA, 20 bp, Artificial Sequence
  Synthetic
acacgagagtaaacgtaaaa